(19) United States
(12) Patent Application Publication
(10) Pub. No.: US 2026/0311877 A1
(43) Pub. Date: Oct. 8, 2026

(54) MODEL FOR MONITORING MINIMAL RESIDUAL DISEASE IN HUMAN HEMATOLOGIC MALIGNANCIES

(71) Applicants: Genetron Health (Beijing) Co., Ltd., Beijing (CN); Beijing Genetron Biotechnology Co., Ltd., Beijing (CN)

(72) Inventors: Xiaochuan LIU, Beijing (CN); Yan WANG, Beijing (CN); Xia ZHONG, Beijing (CN); Yufei YANG, Beijing (CN); Yongfang DONG, Beijing (CN); Yuling WU, Beijing (CN); Zexiong NIU, Beijing (CN); Sizhen WANG, Beijing (CN)

(73) Assignees: Genetron Health (Beijing) Co., Ltd., Beijing (CN); Beijing Genetron Biotechnology Co., Ltd., Beijing (CN)

(21) Appl. No.: 19/571,899

(22) Filed: Mar. 19, 2026

Related U.S. Application Data
(63) Continuation of application No. PCT/CN2025/109543, filed on Jul. 21, 2025.

(30) Foreign Application Priority Data
Apr. 3, 2025 (CN) 202510412727.9

(51) Int. Cl. G16H 50/20; G16B 20/10; G16B 30/10; G16B 40/30; G16H 50/70

(52) U.S. Cl. CPC G16H 50/20; G16B 20/10; G16B 30/10; G16B 40/30; G16H 50/70

(57) ABSTRACT

A model for monitoring minimal residual disease in human hematologic malignancies includes: a data acquisition module, configured to receive pre-treatment and post-treatment DNA sequencing data; a nucleated cell count acquisition module, configured to acquire a nucleated cell count; a clonotype profile and sequence acquisition module, configured to acquire a clonotype profile and a corresponding sequence; a fusion genotype and sequence acquisition module, configured to acquire a fusion genotype and a corresponding sequence; a calculation module, configured to calculate a clonal frequency, a clonal cell abundance, a fusion frequency, and a fusion cell abundance, and determine a dominant clonotype and a dominant fusion type based on the clonal frequency, the clonal cell abundance, the fusion frequency, and the fusion cell abundance; and an MRD calculation and determination module, configured to calculate an MRD value, and determine residual status of a hematologic malignancy based on the MRD value.

### CROSS-REFERENCE TO RELATED APPLICATIONS

This application is a continuation of International Application No. PCT/CN2025/109543, filed on Jul. 21, 2025, which claims priority to Chinese Patent Application No. 202510412727.9, filed on Apr. 3, 2025. The disclosures of the above-mentioned applications are hereby incorporated by reference in their entireties.

### REFERENCE TO AN ELECTRONIC SEQUENCE LISTING

This application contains a sequence listing, which has been submitted electronically as a.xml file and is herein incorporated by reference in its entirety. Said.xml file, created on Mar. 18, 2026, is named “Genetron Health (Beijing) Co, Ltd.xml” and is 164,203 bytes in size.

### TECHNICAL FIELD

This application relates to the technical field of biotechnology, and in particular, to a model for monitoring minimal residual disease (MRD) in human hematologic malignancies.

### BACKGROUND

Hematologic malignancies are common malignant tumors with a high incidence rate and a high mortality rate, posing a severe threat to human health. With the advent of the era of individualized precision therapy for hematologic malignancies, MRD detection for hematologic malignancies has played an increasingly important role in precise monitoring of tumor cells or clones, early intervention, and assessment of immune reconstitution.

MRD means a small number of cancer cells that remain in the body after cancer treatment, and may lead to disease relapse. As a routine detection method for acute lymphoblastic leukemia (ALL), MRD is currently the most important prognostic indicator, and may also serve as a biomarker for monitoring chemotherapy efficacy and drug resistance. Existing MRD detection methods include multiparameter flow cytometry (MFC), PCR-based assays, and next-generation sequencing (NGS). MFC detection uses multicolor flow cytometry to trace abnormal immunophenotypes, and is characterized by a short turnaround time and wide applicability. However, the MFC detection lacks stable sensitivity, relies on professional interpretation of experienced specialists, with antigen phenotypes being prone to change after chemotherapy, thereby limiting the ability of the MFC in early prediction. NGS is an emerging high-sensitivity detection method that uses consensus primers to amplify rearranged fragments of immunoglobulin and T-cell receptor genes, and can detect clonal evolution and trace post-treatment MRD without requiring patient-specific customization. Through efficient bioinformatic analysis, NGS can accurately identify specific clonal sequences, achieving a sensitivity of up to 10-5 to 10-6. Nevertheless, the NGS technique also faces challenges and limitations, including the complexity of a library preparation process, limited sensitivity for ctDNA, tumor spatiotemporal heterogeneity, limited independent prognostic value of IGK-and IGL-based MRD, and high cost. Conventional NGS-based MRD detection techniques typically employ multi-step amplification and pooled library preparation. These approaches also exhibit some drawbacks in practical use, such as a cumbersome library preparation process, the need for two rounds of PCR amplification, a large quantity of samples required, susceptibility to inter-sample contamination, and high cost of library preparation.

### SUMMARY

To solve the above problems, this application provides a model for monitoring minimal residual disease in human hematologic malignancies.

To achieve the above objective, this application puts forward the following technical solutions.

This application provides a model for monitoring minimal residual disease in human hematologic malignancies. The model includes: a data acquisition module, configured to receive pre-treatment and post-treatment DNA sequencing data of the same test subject; a nucleated cell count acquisition module, configured to align sequencing data of a housekeeping gene to a reference genome to acquire a nucleated cell count; a clonotype profile and sequence acquisition module, configured to align the DNA sequencing data to an immune database using an alignment algorithm, and then acquire a clonotype profile and a corresponding sequence using a clustering algorithm, where the immune database includes IGH, IGK, and IGL; a fusion genotype and sequence acquisition module, configured to align the DNA sequencing data to a reference gene sequence to acquire a fusion genotype and a corresponding sequence; a calculation module, configured to calculate a clonal frequency, a clonal cell abundance, a fusion frequency, and a fusion cell abundance based on the nucleated cell count, the clonotype profile and the corresponding sequence, and the fusion genotype and the corresponding sequence, and determine a dominant clonotype and a dominant fusion type based on the clonal frequency, the clonal cell abundance, the fusion frequency, and the fusion cell abundance; and an MRD calculation and determination module, configured to screen a qualified clonotype and a qualified fusion type based on clonotypes and fusion types obtained from the same subject before and after treatment, calculate an MRD value based on a clonal cell abundance and a fusion cell abundance that correspond to the qualified clonotype and the qualified fusion type, and determine residual status of a hematologic malignancy based on the MRD value.

Further, the clustering algorithm includes: calculating an edit distance between sequences based on the DNA sequencing data; performing cluster analysis on sequencing reads based on the edit distance, a relationship between the edit distance and V, D, and J regions of IGH, a relationship between the edit distance and V and J regions of IGK, and a relationship between the edit distance and V and J regions of IGL, so as to acquire a clonotype profile.

Further, a calculation method for the edit distance is:

$$editdist ( i , j ) = MAX \{ \begin{matrix}editdist ( i - 1 , j - 1 ) + m ( i , j ) \\ editdist ( i - 1 , j ) + gap ( i , j ) \\ editdist ( i , j - 1 ) + gap ( i , j ) \\ editdist ( i - 1 , j - 1 ) + S\end{matrix}$$

$$m ( i , j ) = \{ \begin{matrix}ms , & if seq {1}_{i} = seq {2}_{j} \\ ss , & if seq {1}_{i} \neq seq {2}_{j}\end{matrix}$$

$$gap ( i , j ) = \{ \begin{matrix}os , & if gap opening \\ es , & if gap extending\end{matrix}$$

In the formulas above, seq1 and seq2 represent 2 sequences, i represents the ith base in seq1, j represents the jth base in seq2, both i and j start from 1 up to the last base, and initial values thereof are all set to 0; ms represents a match score; when the ith base of seq1 matches the jth base of seq2, m(i, j) is a positive value; ss represents a mismatch score; when the ith base of seq1 does not match the jth base of seq2, m(i, j) is a negative value; os represents a gap-opening score; gap opening represents a start of a gap in sequence alignment; es represents a gap-extending score; gap extending represents continuation of a gap in sequence alignment; S represents a clip score; when the base of seq1 and the base of seq2 are so mismatched that a negative score is excessive, clipping is applied.

Further, the cluster analysis includes: determining an optimal alignment based on an alignment result and the edit distance, and clustering sequencing reads with identical V and J regions of IGH, IGK, and IGL; clustering sequencing reads with identical D and J regions of IGH when IGH-DJ rearrangement occurs; clustering sequencing reads with an identical V region of IGK and KDE element when IGK-KDE rearrangement occurs; clustering sequencing reads within each VJ class based on consistency of CDR3 regions; clustering sequencing reads within each DJ class based on consistency of 50-bp regions centered on a D-J junction of IGH; clustering sequencing reads within each KDE-V class based on consistency of 50-bp regions centered on a junction between an IGK V region and a KDE element, to obtain a sequencing read count corresponding to each subclass; clustering each subclass using a hierarchical clustering method, where a child node and a parent node within each cluster possess highly similar sequences, and the parent node possesses a significantly higher sequencing read count than the child node; summing sequencing read counts of all nodes in each cluster, and reporting a root-node sequence as a distinct clonotype; and combining all reported distinct clonotypes to form a clonotype profile.

Further, a calculation method for the clonal frequency and the clonal cell abundance is:

$$cfl = \frac{{count}_{1}}{{count}_{IGL}}$$

$$cfh = \frac{{count}_{2}}{sum ( {count}_{IGH} , {count}_{HDJ} )}$$

$$cfk = \frac{{count}_{3}}{sum ( {count}_{IGK} , {count}_{KDE} )}$$

$$clonal cell abundance = \frac{clonal cell count}{nucleated cell count}$$

In the formulas above, cfl represents a clonal frequency of a single IGL clonotype, count1 represents a sequence count of a single IGL clonotype, countIGL represents a sequence count of all IGL clonotypes, cfh represents a clonal frequency of a single IGH clonotype, count2 represents a sequence count of a single IGH clonotype, countIGH represents a sequence count of all IGH clonotypes except an IGH-DJ clonotype, countHDJ represents a sequence count of all IGH-DJ clonotypes; cfk represents a clonal frequency of a single IGK clonotype, count3 represents a sequence count of a single IGK clonotype, countIGK represents a sequence count of all IGK clonotypes except an IGK-KDE clonotype, countKDE represents a sequence count of all IGK-KDE clonotypes, and the clonal cell count is a sequence count of a single clonotype.

Further, a calculation method for the fusion frequency and the fusion cell abundance is:

$$fusion frequency = max ( \frac{fc}{hc} , \frac{fc}{bc} )$$

$$fusion cell abundance = \frac{fusion cell count}{nucleated cell count}$$

In the formulas above, fc is a sequence count of a single fusion type, hc represents a total sequence count of IGH, bc is a total sequence count of BCL1 or BCL2, and the fusion cell count represents a sequence count of a single fusion type.

Further, screening criteria for dominant clonotypes are as follows: (1) clonal frequency≥0.03; (2) clonal cell abundance ≥0.002; and (3) clonal frequency distribution is discontinuous, as determined based on the following criteria: (a) clonotypes with a frequency less than 0.03 are selected and arranged in descending order; (b) 10 times a clonal frequency of a 5th clonotype arranged sequentially is used as a threshold; and (c) clonal frequencies of all clonotypes are compared with the threshold, and a clonotype with a clonal frequency greater than or equal to the threshold is considered to satisfy discontinuity of clonal frequency distribution.

Further, screening criteria for dominant fusion types are as follows: (1) fusion frequency ≥0.1%; and (2) fusion cell abundance ≥0.02%.

Further, a calculation method for MRD is:

MRDclone=max(sum(IGH clonal cell abundance), sum(IGK clonal cell abundance)+sum(IGL clonal cell abundance))

$$MRD = max ( {MRD}_{fusion} , {MRD}_{clone} )$$

$$Or$$

$$MRD = {MRD}_{fusion} + {MRD}_{clone} .$$

Further, the model includes: acquiring clonotypes of healthy donors and/or patients with nonhomologous diseases, and calculating a clonal cell abundance of the clonotypes; and excluding, from dominant clonotypes obtained based on pre-treatment DNA sequencing data, those clonotypes that: (i) are non-rarely existent in the healthy donors and/or patients with nonhomologous diseases, and (ii) have a clonal cell abundance greater than or equal to 10-7, where the non-rarely existent clonotypes are clonotypes existent in 5% or more of a population. This step constitutes a basis for subsequent MRD calculation.

The technical solutions provided herein bring at least the following beneficial effects.

This application discloses a simplified library preparation method, in which a sequencing library is constructed in one step by performing only one round of PCR amplification, thereby significantly shortening the library preparation time. Meanwhile, this application employs a single-pool amplification strategy, and mixes primers for IGH, IGK, IGL, and KDE, as well as for IGH-BCL1 and IGH-BCL2 fusion loci, into one pool, thereby enabling detection of both immune receptor rearrangements and key fusion gene events synchronously in a single PCR reaction. This greatly reduces the number of required samples, and is particularly beneficial for tracing post-treatment sample rearrangements. By introducing IGH-BCL fusion detection, this application also further strengthens the ability to capture specific molecular markers for hematologic malignancies such as lymphoma. In terms of construction of a detection system, this technical solution uses hundreds of highly specific primers, provides comprehensive coverage of all immune cell receptor chains (including IGH, IGL, IGK, IGH-DJ, IGK-KDE, and the like) and IGH-BCL fusion hotspot regions, and overcomes the detection blind spots of conventional custom panels. By combining an optimized dominant clone screening algorithm with a uniqueness index assessment model, this application can accurately distinguish malignant clones from physiological rearrangements, thereby effectively addressing the clinical challenge of limited prognostic value for IGK/IGL detection. Experimental validation shows that a limit of detection (LOD) of this technical solution is as low as 10-7, and stable detection performance can still be maintained even in trace samples. In MRD detection, ctDNA sensitivity is a key challenge. ctDNA accounts for only 0.01% of cfDNA and is affected by various factors such as tumor type and burden. The technical solution disclosed herein exhibits excellent detection performance, effectively solves the problem of low ctDNA abundance, and provides accurate MRD detection. This method not only improves detection accuracy but also provides clinicians with more comprehensive and in-depth tumor biology information.

### BRIEF DESCRIPTION OF THE DRAWINGS

To describe the technical solutions in embodiments of this application more clearly, the accompanying drawings to be used in the description of the embodiments are outlined below. Evidently, the drawings outlined below illustrate merely some embodiments of this application, and a person skilled in the art may derive other drawings from such drawings without making any creative effort. In the drawings:

FIG. 1 is a drawing of a relationship between a sample count and a housekeeping gene ratio according to an embodiment of this application, where the horizontal axis represents the sample count and the vertical axis represents a ratio of corrected sequence counts of housekeeping gene 1 to housekeeping gene 2;

FIG. 2 is a schematic diagram of a purified DNA quantification result according to an embodiment of this application;

FIG. 3 is a schematic diagram of an optimal alignment configuration for seq1 and seq2 according to an embodiment of this application;

FIG. 4 is a schematic diagram of layered clusters according to an embodiment of this application;

FIG. 5 is a schematic diagram of a DNA quantification result after purification within a linear range of an ALL cell line (20 μg input) according to Embodiment 3 of this application;

FIG. 6 is a schematic diagram of a DNA quantification result after purification within a linear range of an CLL cell line (20 μg input) according to Embodiment 3 of this application; and

FIG. 7 is a schematic diagram of a DNA quantification result after purification within a linear range of an MM cell line (20 μg input) according to Embodiment 3 of this application.

### DETAILED DESCRIPTION OF THE EMBODIMENTS

The following describes this application in more detail with reference to specific embodiments. A person skilled in the art understands that the following embodiments are merely intended to illustrate this application, but not to limit the scope of this application. Unless techniques or conditions are otherwise expressly specified in an embodiment hereof, the techniques or conditions described in the literature in this field or in an instruction manual of the product are applicable in the embodiment. A reagent or instrument used herein without specifying a manufacturer is a conventional product that is commercially available in the market.

As used herein, the terms “comprise”, “include”, “have”, or any other variations thereof are intended to cover non-exclusive inclusion. For example, a process, method, article, or device that includes listed elements is not limited to such elements, but may include other elements that are not expressly listed or that are inherent to such process, method, article, or device. Unless otherwise expressly specified in the context, a noun preceded by an indefinite article “a” or “an” or a definite article “the” includes a plurality of objects under discussion.

Unless otherwise defined, all technical and scientific terms used herein bear the same meanings as typically understood by a person skilled in the art. This application may be implemented not only by using the specific methods, devices, and materials described in embodiments of this application, but also by using any existing methods, devices, and materials in the prior art similar or equivalent to the methods, devices, and materials described in an embodiment of this application based on existing knowledge of those skilled in the art and the description of this application.

Unless otherwise specified, the experimental methods, detection methods, and preparation methods disclosed in this application all employ techniques in molecular biology, immunology, laboratory medicine, gene sequencing technology, and bioinformatics technology in this technical field and conventional techniques in related fields.

An embodiment of this application provides a model for monitoring minimal residual disease in human hematologic malignancies. The model includes:
(1) a data acquisition module, configured to receive pre-treatment and post-treatment DNA sequencing data of the same test subject;
(2) a nucleated cell count acquisition module, configured to align sequencing data of a housekeeping gene to a reference genome to acquire a nucleated cell count;
(3) a clonotype profile and sequence acquisition module, configured to align the DNA sequencing data to an immune database using an alignment algorithm, and then acquire a clonotype profile and a corresponding sequence using a clustering algorithm, where the immune database includes IGH, IGK, and IGL;
(4) a fusion genotype and sequence acquisition module, configured to align the DNA sequencing data to a reference gene sequence to acquire a fusion genotype and a corresponding sequence;
(5) a calculation module, configured to calculate a clonal frequency, a clonal cell abundance, a fusion frequency, and a fusion cell abundance based on the nucleated cell count, the clonotype profile and the corresponding sequence, and the fusion genotype and the corresponding sequence, and determine a dominant clonotype and a dominant fusion type based on the clonal frequency, the clonal cell abundance, the fusion frequency, and the fusion cell abundance; and
(6) an MRD calculation and determination module, configured to screen a qualified clonotype and a qualified fusion type based on clonotypes and fusion types obtained from the same subject before and after treatment, calculate an MRD value based on a clonal cell abundance and a fusion cell abundance that correspond to the qualified clonotype and the qualified fusion type, and determine residual status of a hematologic malignancy based on the MRD value.

Specifically, the screening for qualified clonotypes and fusion types may include:
(1) clonotypes and fusion types that exist in the same test subject after treatment and that are identical to the pre-treatment dominant clonotypes and dominant fusion types; and
(2) clonotypes that exist in the same test subject in subsequent tests and that are identical to the newly diagnosed post-treatment dominant clonotypes.

The newly diagnosed post-treatment dominant clonotypes refer to those dominant clonotypes that emerge after treatment and are not identical to any pre-treatment dominant clonotypes. In subsequent detections, the dominant clonotypes newly diagnosed after treatment are tracked.

In MRD detection, the sensitivity of ctDNA is a key challenge. ctDNA accounts for only 0.01% of cfDNA and is affected by various factors such as tumor type and burden. Especially in early-stage cancer patients, low-abundance ctDNA may lead to false negatives for MRD. Based on a BCR database, whether a tumor is a cancer cell clone can be determined through dominant clone screening and uniqueness index assessment, thereby solving the problem of limited prognostic value of IGK and IGL-MRD. This method not only improves detection accuracy but also provides clinicians with more comprehensive and in-depth tumor biology information, exhibiting excellent detection performance, and achieving a limit of detection as low as 10-7.

Specifically, the data acquisition module coordinates with a monitoring device through a testing kit to obtain pre-treatment and post-treatment DNA sequencing data of a plurality of samples of the same test subject. The terms “same clonotype” and “same fusion type” refer to those with identical gene sequence.

Specifically, the testing kit includes: a forward outer primer F1, a forward inner primer F2, and a reverse outer primer R. The forward outer primer F1 is formed of a sequencing adapter sequence 1, a barcode sequence, and a forward universal sequence 1. The adapter sequence 1 is shown in SEQ ID No. 5, the barcode sequences are shown in SEQ ID No. 6-15, and the forward universal sequence 1 is shown in SEQ ID No. 16.

The forward inner primer F2 is formed of a forward universal sequence 2 and a forward specific primer sequence. The forward universal sequence 2 is shown in SEQ ID No. 17, and the forward specific primer sequences are shown in SEQ ID No. 18-163.

The reverse outer primer R is formed of a reverse specific primer sequence, a reverse adapter sequence, and a reverse sequencing adapter sequence, and is phosphorylated at the 5′ end of the primer. The reverse specific primer sequences are shown in SEQ ID No.164-183, the reverse adapter sequence is shown in SEQ ID No.184, and the reverse sequencing adapter sequence is shown in SEQ ID No.185.

Optionally, the kit includes 2 housekeeping genes. The forward specific primer sequence of the first housekeeping gene is shown in SEQ ID No. 1, and the reverse specific primer sequence of the first housekeeping gene is shown in SEQ ID No. 2. The forward specific primer sequence of the second housekeeping gene is shown in SEQ ID No. 3, and the reverse specific primer sequence of the second housekeeping gene is shown in SEQ ID No. 4.

### I. Selection of Housekeeping Genes

Specifically, considering diversity of sample types, 2 amplicons are selected as housekeeping genes to ensure stability of the total sequence count of the housekeeping genes. For selecting the housekeeping genes, regions with high sequence specificity are selected preferentially for design. Furthermore, due to a relatively long interval between a pre-treatment sample and a post-treatment sample, 3 SNP loci are specifically designed on the housekeeping genes amplicon to ensure traceability and comparison of the same sample before and after treatment. Each locus has 3 genotypes. Samples collected from the same individual at different time periods exhibit the same genotype at the 3 SNP loci, thereby significantly reducing the risk of sample confusion during sampling and experimental procedures.

2 genes are selected as housekeeping genes, with hg19 as a reference. Specific information is as follows:

### (1) Housekeeping Gene 1

Location information is chr20: 15124894-15125033; the forward specific primer sequence is GGCCTACAATTCAAATTAATGTAAAAACTGC (SEQ ID No:1); the location of the forward primer is chr20: 15124894-15124924; the location of the reverse primer is chr20: 15125006-15125033; the reverse specific primer sequence is TCTGATTCAAACTTTTCTTTTGTGGCTG (SEQ ID No:2).

### (2) Housekeeping Gene 2

Location information is chr5: 17374840-17374979; the forward specific primer sequence is GCACTTTCAATCAACTGTGTTAGATTGA (SEQ ID No:3); the forward primer location is chr5: 17374840-17374867; the reverse primer location is chr5: 17374951-17374979; the reverse specific primer sequence is TATGATCCACATTGTATGGTTTTTAGGCA (SEQ ID No: 4).

The primers of the housekeeping genes exhibit good sequence specificity on the genome, without amplification of pseudogene sequences. After whole-genome alignment, the housekeeping gene 1 and the housekeeping gene 2 each contain a 140-bp fragment that aligns 100%, with the location as expected. The other two well-aligned fragments are merely partial regions of the amplicons, and are not longer than 30 bp and are considered highly unlikely to be amplified, as detailed in Table 1.

TABLE 1
Results of housekeeping genes alignment

| | House- | House- | House- | House- | House- | House- |
|---|---|---|---|---|---|---|
| | keeping | keeping | keeping | keeping | keeping | keeping |
| | gene 1 | gene 1 | gene 1 | gene 2 | gene 2 | gene 2 |
| Amplicon start | 1 | 44 | 119 | 1 | 8 | 47 |
| position | | | | | | |
| Amplicon end | 140 | 82 | 138 | 140 | 36 | 66 |
| position | | | | | | |
| Consistency | 100.0% | 65.4% | 100.0% | 100.0% | 77.0% | 100.0% |
| Chromosome | chr20 | chr10 | chr18 | chr5 | chr2 | chr2 |
| Strand | Positive | Positive | Negative | Positive | Positive | Negative |
| Start position | 15124894 | 69208081 | 48091844 | 17374840 | 149931841 | 22875728 |
| End position | 15125033 | 69208106 | 48091863 | 17374979 | 14993186 | 228757 |
| Span (bp) | 140 | 26 | 20 | 140 | 26 | 20 |
| Target fragment | Yes | No | No | Yes | No | No |

The housekeeping genes contains 3 SNP loci, as shown in Table 2. These loci stably exist in both normal cells and tumor cells, and are independent of the cell cycle and cell activation status without being affected by any endogenous or exogenous factors (such as experimental treatments), and can serve as alignment information for pre-treatment and post-treatment samples, thereby preventing sample contamination.

TABLE 2
Housekeeping genes SNP locus information

| | | | | | 1,000-person |
|---|---|---|---|---|---|
| | dbSNP | | Reference | Mutant | genome |
| | ID | Position | base | base | frequency |
| House- | rs403365 | chr20: | C | T | C = 0.415935 |
| keeping | | 15125000 | | | T = 0.584065 |
| gene 1 | | | | | |
| House- | rs445251 | chr20: | G | C | G = 0.415535 |
| keeping | | 15124933 | | | C = 0.584465 |
| gene 1 | | | | | |
| House- | rs159606 | chr5: | A | G | A = 0.343850 |
| keeping | | 17374898 | | | G = 0.656150 |
| gene 2 | | | | | |

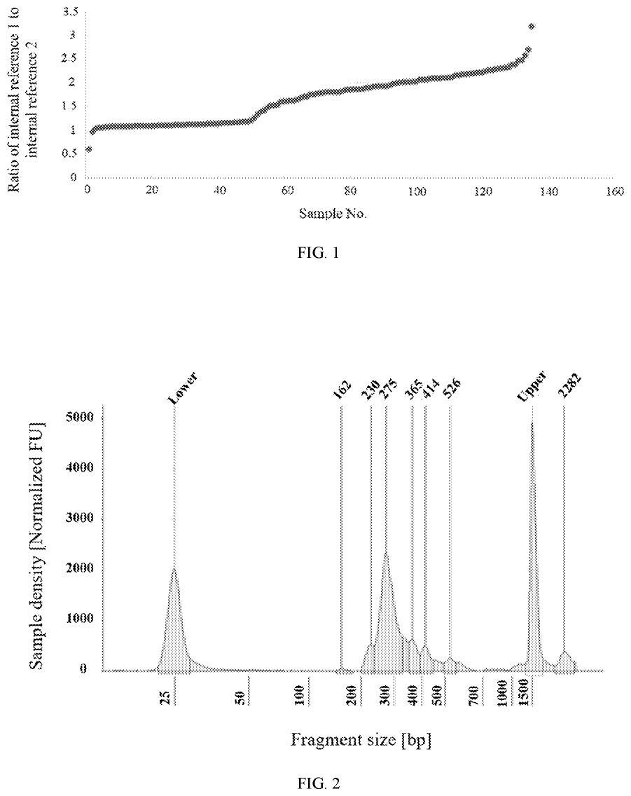

136 clinical hematological malignancy samples are selected for performing library construction, purification, quantification, sample pooling and dilution, sequencing, and data analysis according to the following steps. The ratio of the sequence count of the housekeeping gene 1 to the sequence count of the housekeeping gene 2 is calculated, as shown in FIG. 1. The results show that the sequence count ratio corrected by the two housekeeping genes remains relatively stable.

### II. Library Preparation

Different samples use different specific adapters. gDNA from the same sample is mixed to form one pool, and uses the same specific adapter (forward outer primer). Samples are divided into a pre-treatment group and a post-treatment group, and the two groups differ in the initial amount of gDNA.

The gDNA from each clinical sample is aliquoted into separate tubes to construct libraries, and each tube contains the same amount of gDNA. 0.2 mL PCR tubes, the number of which is equivalent to the number of clinical samples plus 1 (control sample), are taken to formulate a reaction system according to Table 3. The ratio between the forward outer primer, the forward inner primer mixture, and the reverse primer mixture is 5:3: 20.

TABLE 3
Amplification system

| Reagent | Volume (μL) |
|---|---|
| SuperPlex ™ Premix | 15 |
| Genome DNA of a sample | Pre-treatment sample 200 ng |
| | Post-treatment sample 2-20 μg |
| Forward outer primer F1 (100 μM) | 0.5 |
| Forward inner primer MIX1 (100 μM) | 0.3 |
| Reverse primer MIX2 (100 μM) | 2 |
| DNAase-free H2O | Fill to 30 μL |

Primer mixture formulation:

A primer sequence is formed of a forward outer primer F1, a forward inner primer F2, and a reverse outer primer R, as shown in Table 4 and Table 5.

The forward outer primer F1 is formed of a sequencing adapter sequence 1 (CAAGCAGAAGACGGCATACGAGAT SEQ ID No:5), a barcode sequence (SEQ ID No:6-15), a forward universal sequence 1 (GGCACCCGAGAATTCCA SEQ ID No:16).

The forward inner primer F2 (that is, the housekeeping gene forward primer, IGH-V forward primers, IGH-D forward primers, IGK-V forward primers, IGKED forward primer, IGL-V forward primers, fusion forward primers) is formed of a forward universal sequence 2 (CGAACCGCTTTCGGAGG SEQ ID No:17) and a forward specific primer sequence (SEQ ID No: 18-163).

The reverse outer primer R (that is, the housekeeping gene reverse primer, the IGH-J reverse primers, IGK-J reverse primers, the IGKDE reverse primer, and the IGL-J reverse primers) is formed of a reverse specific primer sequence (SEQ ID No:164-183), a reverse adapter sequence (TCAGAGTTCTACAGTCCGACGATC SEQ ID No:184), and a reverse sequencing adapter sequence (AATGATACGGCGACCACCGAGATCTACACGT SEQ ID No:185), and is phosphorylated at the 5′ end of the primer.

Each forward outer primer F1 is diluted to 100 μM separately.

Each forward inner primer F2 is diluted to 100 μM separately, and mixed in equal amounts of substance to obtain a forward inner primer MIX1 (that is, a mixture of a housekeeping gene forward primer, IG forward primers, and fusion forward primers).

The reverse outer primer R is diluted to 100 μM separately, and mixed in equal amounts of substance to obtain a reverse primer MIX2 (that is, a mixture of a housekeeping gene reverse primer and IG reverse primers).

The PCR amplification reaction conditions are shown in Table 6.

TABLE 4
Barcode sequences information

| Primer name | Primer sequence |
|---|---|
| Barcode sequence 1 | TAGGTCCGAT (SEQ ID No: 6) |
| Barcode sequence 2 | GGACGGAATC (SEQ ID No: 7) |
| Barcode sequence 3 | CTTACTGCCG (SEQ ID No: 8) |
| Barcode sequence 4 | ACCTAATTGA (SEQ ID No: 9) |
| Barcode sequence 5 | TTCGTATCCG (SEQ ID No: 10) |
| Barcode sequence 6 | GGTAACGAGC (SEQ ID No: 11) |
| Barcode sequence 7 | CAACGTATAA (SEQ ID No: 12) |
| Barcode sequence 8 | ACGTCGCGTT (SEQ ID No: 13) |
| Barcode sequence 9 | TTCTGCTAGC (SEQ ID No: 14) |
| Barcode sequence 10 | AGGAAGATAG (SEQ ID No: 15) |

TABLE 5
Specific primer sequences information

| | | | SEQ ID |
|---|---|---|---|
| Primer name | Amplicon | Primer sequence | No |
| Forward specific | IGH-V | AAGGGCTTGAGTGGATGGGA | 18 |
| primer | IGH-V | AACATGGACCCTGTGGACAC | 19 |
| | IGH-V | ACAGCCTACATGGAGCTGAGC | 20 |
| | IGH-V | ACCTTGAAGGAGTCTGGTCCT | 21 |
| | IGH-V | AGTCACCATGACCAGGGACA | 22 |
| | IGH-V | TGCAGCTGCAGGAGTCG | 23 |
| | IGH-V | GGAAAGGCYTGGAGTGGATG | 24 |
| | IGH-V | CCCTGAAGCTGAGCTCTGTG | 25 |
| | IGH-V | GGTGAAGCCCTCGCAGAC | 26 |
| | IGH-V | CCATCGAGAGGCCTTGAGTG | 27 |
| | IGH-V | AAGTCGAATAACCATCAACCCAGAC | 28 |
| | IGH-V | CCCTGAGACTCTCCTGTGCAG | 29 |
| | IGH-V | CGGGGAGTCTCTGARGATCTCC | 30 |
| | IGH-V | CTGCAGTGGAGCAGCCT | 31 |
| | IGH-V | CCGATTCACCATCTCCAGAGAC | 32 |
| | IGH-V | ATGCACTGGGTCCGCCA | 33 |
| | IGH-V | CTTGGTCCAGCAGGCTTCA | 34 |
| | IGH-V | AGGGAAGGGGCTGGAGT | 35 |
| | IGH-V | CCAGGGAACAGACTGGAGT | 36 |
| | IGH-V | CTGGGAAAGGGCTGGAGTAG | 37 |
| | IGH-V | CTTCACAACAGGAATTGGTGTGG | 38 |
| | IGH-V | CTTCACAACAGGGATAGGTGTGG | 39 |
| | IGH-V | CCAGGGAAGGGAATGGAGT | 40 |
| | IGH-V | TCTGGGAGGGAATGGAGTGG | 41 |
| | IGH-V | GGCAACAGGCTGGAATGG | 42 |
| | IGH-V | AAGGAAGGGACTGGAGCGA | 43 |
| | IGH-V | CCAGTCCTCAGTCAAGGGAGT | 44 |
| | IGH-V | CCAGGGAAGAAGCTGAAGTGG | 45 |
| | IGH-V | GCTTCAAGAAAGGGGCTGGA | 46 |
| | IGH-V | CCTGGATCCACCATCTCCCA | 47 |
| | IGH-V | GGCAAGGGGCTGGAGTGA | 48 |
| | IGH-V | GCTTGCCCCTAGAAGATGGACA | 49 |
| | IGH-V | GAAGGCGCTGGGGCAC | 50 |
| | IGH-V | GGGACAGAGGCTGGAGTG | 51 |
| | IGH-V | CTGTAGGCACTGGATGGAAAGC | 52 |
| | IGH-V | CATGGGAGGTACTGGAGTGG | 53 |
| | IGH-V | CAGGGAAGGGGCTGGAATG | 54 |
| | IGH-V | CAGGGAATGGGCTGGAGTT | 55 |
| | IGH-V | AGGAAAGGGGCTGCAGTGA | 56 |
| | IGH-V | AGGAGAGGGGCTGGAGTG | 57 |
| | IGH-V | TTCATGGCAAGTGTTGGTGAGG | 58 |
| | IGH-V | CTCAGTGAAGGTCTCCTGCAAG | 59 |
| | IGH-V | CTTCTGGGAAAGGGCTGGAGTGG | 60 |
| | IGH-V | TTCAGGACAGGGGTTGGTGT | 61 |
| | IGH-V | CCTGGACAGGGCTTTGAGTGG | 62 |
| | IGH-V | CCATGGACAAGGGCTTGAGGAA | 63 |
| | IGH-V | ATGACAGGGGTGGGTGT | 64 |
| | IGH-V | CCAGGGAAGGGACTGGAGT | 65 |
| | IGH-V | AATGAGACTCTAGGGAAGGGGC | 66 |
| | IGH-V | CACATGGGAAGGACTGGAGT | 67 |
| | IGH-V | ACTAGCCCGCTTCCATGG | 68 |
| | IGH-V | CTCCAGGAAAGGGTTTGGAGTGG | 69 |
| | IGH-V | GAAGGCCCTGGAGTGGCTT | 70 |
| | IGH-V | CCCTGGACAAGGGCTTGAAAGG | 71 |
| | IGH-V | GGGCTGGAGTGGGTAGGTTTCA | 72 |
| | IGH-V | GGAAGGGTCTGGAGTGGGTATCAG | 73 |
| | IGH-V | AAGGGGCTGGAGTGGGTATC | 74 |
| | IGH-V | GAGCAGCCTGAGATCTGAGGACT | 75 |
| | IGH-V | CCGMTTCATCATCTCCAGAGAC | 76 |
| | IGH-D | GATTCYGAACAGCCCCGAGTCAC | 77 |
| | IGH-D | GGATTTTGTGGGGGYTCGTGTCA | 78 |
| | IGH-D | GGTAGGTTTGRRGTGAGGTCTG | 79 |
| | IGH-D | GCCGGTAGGTTTAGAATGAGGTC | 80 |
| | IGH-D | CTTTTTGTGAAGGSCCCTCCTRCT | 81 |
| | IGH-D | CACTCAGGGGCTTTTTGTGAAGG | 82 |
| | IGH-D | GCCTCCAGTTCCAGGTGTGGT | 83 |
| | IGH-D | GGTTTCTGAAGSTGTCTGTRTCAC | 84 |
| | IGK-V | CCAGGAGACAAAGTCAACATCTCCTG | 85 |
| | IGK-V | CCTCACAATTAATCCTGTGGAAGCT | 86 |
| | IGK-V | AGAGCCGGCCTCCATCT | 87 |
| | IGK-V | AGTGGGGTCCCATCAAGGTTC | 88 |
| | IGK-V | GGGAAAGAGTCACCCTCTCCTG | 89 |
| | IGK-V | CTGGAGTCCCAGACAGGTTCAG | 90 |
| | IGK-V | CAGATTTCACTCTCACCATCAGCAG | 91 |
| | IGK-V | GGAGAGCCATCCTCCATTTCCTG | 92 |
| | IGK-V | GGAAAGAGCCACCCTCTCCTG | 93 |
| | IGK-V | GGCCAGTCAGAGCATTGGTAGTAG | 94 |
| | IGK-V | CCCAGACAGATTCAGTGGCAGTG | 95 |
| | IGK-V | TGGAGAAAAAAGCCACCCTGACTTG | 96 |
| | IGK-V | CTGGAGTGCCAGATAGGTTCAGTG | 97 |
| | IGK-V | GGTTCAGTGGAAGTGGATCTGG | 98 |
| | IGK-V | GAAGGAGCCACCCTCACCTG | 99 |
| | IGK-V | GGAGACAGTGTCACCATCACTTG | 100 |
| | IGK-V | CCATCTCGGTTCAGTGGCAG | 101 |
| | IGK-V | TGGAGTCCCAGACAGGTTCAATAG | 102 |
| | IGK-V | GAGACAGCGTCACCATCACTTG | 103 |
| | IGK-V | ACTGCATCTGTAGGAGAGAGAGTCA | 104 |
| | IGK-V | GAGAAAGTCACCATCACCTGCCA | 105 |
| | IGK-V | CTGGAGAGCCAGCCTCCATC | 106 |
| | IGK-V | TGCATCTGTAGGAGACAGAGTCAG | 107 |
| | IGK-V | CAGGCAGGGCATTAGCCAT | 108 |
| | IGK-V | AGGTTCAGGGGTAATGGGTCAG | 109 |
| | IGK-V | TCTTTATCTCTAGGGGAAAAAGCCACC | 110 |
| | IGK-V | TCCCACCTCGATTCAGTGGC | 111 |
| | IGK-V | GGTTCAGTGGAAGTGGGTCTGG | 112 |
| | IGKDE | CAGCTGCAGACTCATGAGGAG | 113 |
| | IGL-V | GGACCGTGGGACAGAAGGTCA | 114 |
| | IGL-V | GCTTCTCAGGCTCCAGCTCTG | 115 |
| | IGL-V | TGTGGGGCAGGTGGGTAC | 116 |
| | IGL-V | CCTCTCTGCACATCTGAGAAATGC | 117 |
| | IGL-V | GGAGCATGAGCCAGACTTACCTG | 118 |
| | IGL-V | CGGATCAGCCAGACTCTCCTG | 119 |
| | IGL-V | GGAGCAACAGCCAGACTCC | 120 |
| | IGL-V | GTCACCATCTCCTGCACTGG | 121 |
| | IGL-V | CCACAAGGCAGAGGCTCACT | 122 |
| | IGL-V | GGAGGGACAGTCACACTCACTTG | 123 |
| | IGL-V | CCTCGGTCAAGCTCACCTG | 124 |
| | IGL-V | CTCTGGGAGCATCTTCCAGACTC | 125 |
| | IGL-V | GAGCCTCGGTCAAGCTTACCTC | 126 |
| | IGL-V | CTCACCATCTCTGGACTGAAGACTGA | 127 |
| | IGL-V | CTTGAGACAGACCGCCACACT | 128 |
| | IGL-V | CCGCTTCTCTGGATCCAAAGAT | 129 |
| | IGL-V | TCTGCATCTTCTGGAGCATCAGTC | 130 |
| | IGL-V | TTCTCTGGCTCCAAGTCTGGC | 131 |
| | IGL-V | ATCCCTGATCGCTTCTCAGTCTT | 132 |
| | IGL-V | CTCACTGACTGTGTCCCCAG | 133 |
| | IGL-V | TGTCTGTGGCCTTGGGACAA | 134 |
| | IGL-V | ACAGACASCCAGGATCACCTG | 135 |
| | IGL-V | AGGACAGATGACTAGGATCACCT | 136 |
| | IGL-V | TCCTGGACAGTCGATCACCATC | 137 |
| | IGL-V | GTCCCTAGGACAGATGGCCAG | 138 |
| | IGL-V | ACAGATGGCCAGGATCACCT | 139 |
| | IGL-V | CCAGGACAAACGGCCAGGAT | 140 |
| | IGL-V | GACAGAGGGCCAGGATCACCTA | 141 |
| | IGL-V | ACAGGCAGCCATGATCACCTG | 142 |
| | IGL-V | GGTCTCCTGGACAGTCAGTCAC | 143 |
| | IGL-V | CTGGGAGCCTCGATCAAGCTC | 144 |
| | IGL-V | GGACGTACGGCCAGGATCATC | 145 |
| | IGL-V | AGGACAGACAGCCAGCATCA | 146 |
| | IGL-V | ACAGTCAGCCAGGGTAACCTG | 147 |
| | IGL-V | AGGACACTCACTCCCCCAC | 148 |
| | IGL-V | CTTGGGACAGACAGTCAGGATCAC | 149 |
| | IGL-V | GCCTCCCTGGCCATCTCTG | 150 |
| | IGL-V | CGTCTGTCTGTGGCTAAACCAG | 151 |
| | IGL-V | ATGGCATCTCTGACCATCTCTG | 152 |
| | IGL-V | GGATCCCTGAGCGATTCTCTGG | 153 |
| | Fusion | CACTGTCTGGATGCACCGC | 154 |
| | Fusion | ATGACCAGCAGATTCAAATCTATGG | 155 |
| | Fusion | GGAATAACTCTGTGGCATTATTGCA | 156 |
| | Fusion | TGAGCACCTGCTGGATACAACA | 157 |
| | Fusion | CTGGGTGACAGAGCAAAACATGAA | 158 |
| | Fusion | CTGTAATGACTGGGGAGCAAATCTT | 159 |
| | Fusion | GTTGGCGTGGTTTAGAGAGCT | 160 |
| | Fusion | TCCCTTCTGAAAGAAACGAAAGCA | 161 |
| | Fusion | GCATAGAGCAAGCGCCCAATAAATA | 162 |
| | Fusion | CAGAAATGAATGCCATCTCAAATCCAA | 163 |
| Reverse specific | IGH-J | ACCTGAGGAGACGGTGACC | 164 |
| primer | IGH-J | GAGGAGACAGTGACCAGGGT | 165 |
| | IGH-J | CTGAAGAGACGGTGACCATTGT | 166 |
| | IGH-J | ATGTGGCTGCGGTCTCCA | 167 |
| | IGH-J | TGACGTTCCCAGGGAGACG | 168 |
| | IGH-J | CTGACCCCTCAGAAGCCAGA | 169 |
| | IGK-J | ACTTACGTTTAATCTCCAGTCGTGTC | 170 |
| | IGK-J | CGTTTGATCTCCACCTTGGTCCC | 171 |
| | IGK-J | ACGTTTGATATCCACTTTGGTCCC | 172 |
| | IGK-J | GATCTCCAGCTTGGTCCCCT | 173 |
| | IGK-J | ATTTCCACCTTGGTCCCTTGGC | 174 |
| | IGK-J | CGTTTGAATTCTACCCTGCTCCC | 175 |
| | IGK-J | GATSTCCAGTTTGGTCCCCT | 176 |
| | IGK-INTR | GGCTTTGGTGGCCATGC | 177 |
| | IGL-J | CTAGGACGGTGACCTTGGTCC | 178 |
| | IGL-J | CGGTCAGCTTGGTCCCTCCG | 179 |
| | IGL-J | ATGATCAGCTGGGTTCCTCCAC | 180 |
| | IGL-J | AGGACGGTCACCTTGGTGC | 181 |
| | IGL-J | AGGACGGTCAGCTGGGT | 182 |
| | IGL-J | AGACTCATCTAGGACGGTCAGCT | 183 |

TABLE 6
PCR amplification reaction conditions

| | | Cycle |
|---|---|---|
| Temperature | Time | count |
| 95° C. | 3 min | 1 |
| 95° C. | 30 s | 26 |
| 60° C. | 90 s | |
| 72° C. | 90 s | |
| 72° C. | 10 min | 1 |
| 4° C. | ∞ | 1 |

### III. Library Purification

The amplification product obtained in step 2 is purified using an MPure XP-PCR product purification kit (Beckman Coulter, A63882), so as to obtain a purified DNA library. The purified DNA library solution may be stored at 4° C. for up to 16 hours. For longer-term storage, the storage temperature needs to be −18° C. or lower.

### IV. Library Quantification

The concentration and fragment size of the purified DNA library obtained above are quantified using TapeStation 2200/4200. The quantification result obtained using TapeStation 2200 is shown in FIG. 2.

Agilent 2200/4200 quality control: After purification with magnetic beads, the libraries with a target fragment of 220 bp to 400 bp in length and with a proportion of dimer bands (shorter than 200 bp) being not greater than 20% are qualified and can be directly sequenced. If the proportion of the dimers after purification exceeds 20%, the library is unqualified and needs to be reconstructed.

### V. Library Sample Pooling and Dilution

(1) All the DNA libraries selected in step IV are mixed at an appropriate mass ratio, and the mixture is labeled as a sequencing library pool.

(2) A total concentration of target fragments within the 220-400 bp range in the sequencing library pool is defined as a library concentration. It is recommended that a sequencing data volume per sample is 5 gigabytes.

Note: The pooled library needs to be vortexed for 1 minute and centrifuged for 2 seconds. This step needs to be repeated at least 3 times until the pooled library is thoroughly mixed. It is essential to ensure that the library is thoroughly mixed before a quantitative test.

### VI. Sequencing

After library quantification by Qubit, a specified amount of data is loaded onto a GENETRON S2000 platform for sequencing. The data output is raw sequencing data in PE150 fastq files.

By using the data acquisition module in the model for monitoring minimal residual disease of leukemia according to this application, the raw sequencing data is acquired, that is, the pre-treatment and post-treatment DNA sequencing data is received from the same test subject.

### VII. Data Analysis

It needs to be noted that the nucleated cell count acquisition module, which is configured to align the sequencing data (sequencing reads) of the housekeeping genes (chr5: 17374840-17374979, chr20: 15124894-15125033, human hg19 genome location) to the reference genome (hg19, human hg38) to acquire a nucleated cell count, is an existing technology and will not be elaborated here.

A clonotype profile and sequence acquisition module is configured to align the DNA sequencing data to an immune database using an alignment algorithm, and then acquire a clonotype profile and a corresponding sequence using a clustering algorithm, where the immune database includes IGH, IGK, and IGL.

Specifically, the clonotype profile and sequence acquisition module aligns the sequencing data to an immune database using an alignment algorithm(reference document: https://academic.oup.com/nar/article/41/10/e108/1075719), and then acquires a clonotype profile using a clustering algorithm, where the immune database includes IGH, IGK, and IGL (source: IMGT http://www.imgt.org). The clonotype profile includes, but is not limited to, clonotypes of IGH, IGK, IGL, IGH-DJ, and IGK-KDE types or subsets of such types.

The clustering algorithm first calculates an edit distance between sequences, specifically as follows:

$$editdist = f ( seq 1 , seq 2 )$$

In the formula above, editdist represents the edit distance between sequences, and the edit distance is a sequence-related function. seq1 and seq2 represent 2 sequences. The edit distance may be calculated using the following formula:

$$editdist ( i , j ) = MAX \{ \begin{matrix}editdist ( i - 1 , j - 1 ) + m ( i , j ) \\ editdist ( i - 1 , j ) + gap ( i , j ) \\ editdist ( i , j - 1 ) + gap ( i , j ) \\ editdist ( i - 1 , j - 1 ) + S\end{matrix}$$

In the formula above, i represents the ith base in seq1, j represents the jth base in seq2, both i and j start from 1 up to the last base, and initial values thereof are all set to 0.

$$m ( i , j ) = \{ \begin{matrix}ms , & if seq {1}_{i} = seq {2}_{j} \\ ss , & if seq {1}_{i} \neq seq {2}_{j}\end{matrix}$$

ms represents a match score. When the ith base of seq1 matches the jth base of seq2, m(i, j) is a positive value. ss represents a mismatch score. When the ith base of seq1 does not match the jth base of seq2, m(i, j) is a negative value.

$$gap ( i , j ) = \{ \begin{matrix}os , & if gap opening \\ es , & if gap extending\end{matrix}$$

os represents a gap-opening score (a negative value); gap opening represents a start of a gap in sequence alignment; es represents a gap-extending score (a negative value); and gap extending represents continuation of a gap in sequence alignment. Typically, os is greater than es.

S represents a clip score (a negative value). When the base of seq1 and the base of seq2 are so mismatched that the negative value is excessive, clipping is applied.

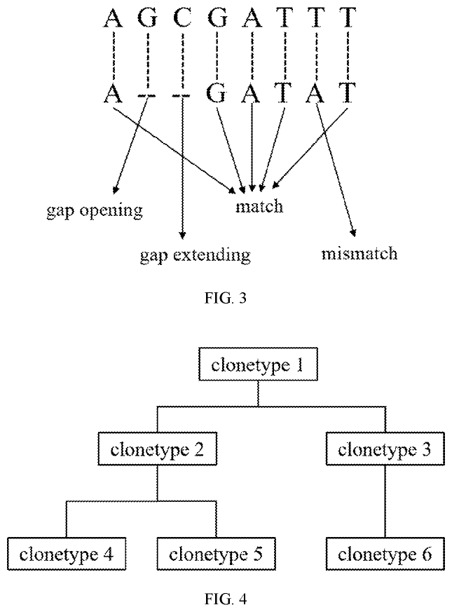

An optimal alignment is obtained, as detailed in FIG. 3.

To facilitate subsequent data clustering, the values of ms, ss, os, es, and S are made to satisfy the following conditions:

$$\{ \begin{matrix}ms > 0 \\ ss < 0 \\ os < 0 \\ es < 0 \\ S < 0 \\ \vert S \vert < \vert os \vert + n * \vert es \vert + 2 \\ \vert os \vert > \vert ss \vert \geq \vert es \vert\end{matrix}$$

In the inequalities above, n is an integer greater than or equal to 0 and less than a sequencing read length. The sixth inequality in the above indicates that a boundary n exists such that the absolute value of a soft-clipping penalty(S) is necessarily less than a gap opening (os) penalty plus n gap extending(es) penalties plus 2.

Preferably, ms may take a value of 1, and other values may be an integer negative multiple of ms.

The above definition enables assignment of weights for various scenarios, thereby facilitating subsequent data clustering, and preventing ambiguous boundaries and parent-node confusion during clustering. For example, ms=1, ss=−4, os=−5, es=−1, and S=−6.

Subsequently, cluster analysis is performed on sequencing reads based on the edit distance, the relationship between the edit distance and V, D, and J regions of IGH, the relationship between the edit distance and V and J regions of IGK, and the relationship between the edit distance and V and J regions of IGL, so as to acquire a clonotype profile. The detailed process is as follows:
(1) Determining an optimal alignment (that is, the alignment with the highest edit distance score) based on result of an alignment algorithm and the edit distance, and clustering the sequencing reads with identical V and J regions of the IGH, IGK, and IGL; clustering the sequencing reads with identical D and J regions of IGH when IGH-DJ rearrangement occurs; and clustering sequencing reads with an identical V region of IGK and the KDE element when IGK-KDE rearrangement occurs.
(2) Clustering the sequencing reads within each VJ class based on consistency of CDR3 regions; clustering sequencing reads within each DJ class based on consistency of 50-bp regions centered on a D-J junction of IGH; and clustering sequencing reads within each KDE-V class based on consistency of 50-bp regions centered on a junction between an IGK V region and a KDE element, to obtain a sequencing read count corresponding to each subclass.
(3) Clustering each subclass using a hierarchical clustering method, where a child node and a parent node within each cluster possess highly similar sequences, and the parent node possesses a significantly higher sequencing read count than the child node.

Specifically, this step includes: obtaining an average sequencing-base error rate, which is generally an empirical constant related to the sequencing platform and the like; and estimating a subclass coefficient (hereinafter referred to as factor, greater than 1) based on the error rate. Sorting the subclasses in each class (VJ class, DJ class, and KDE-V class) in descending order of the sequencing read count. Comparing the high-read-count class with other subclasses successively based on the sorting result and the edit distance algorithm, and performing clustering by using a hierarchical clustering method. A child node and a parent node within each cluster possess highly similar sequences, and the parent node possesses a significantly higher sequencing read count than the child node. That is, the read count of the parent node divided by the read count of the child node is greater than factor).
(4) Summing sequencing read counts of all nodes in each cluster, and reporting a root-node (top parent node) sequence as a distinct clonotype.
(5) Combining all reported distinct clonotypes to form a clonotype profile.

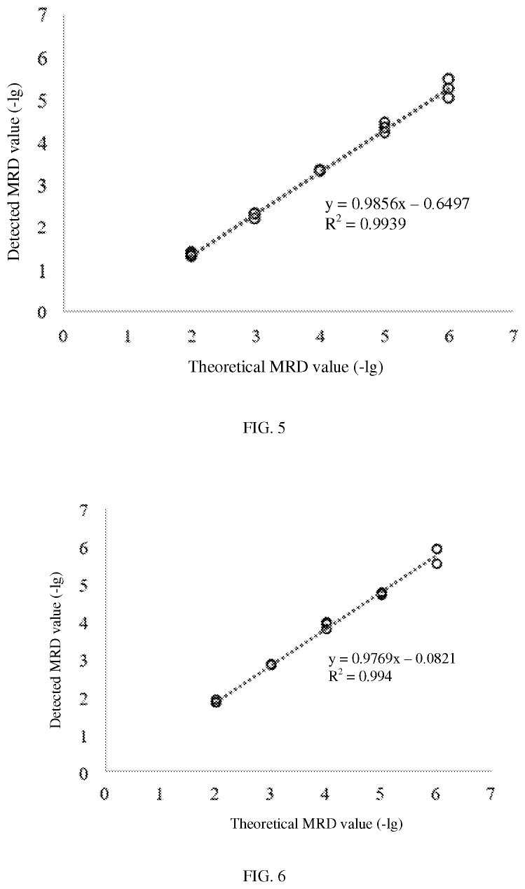

FIG. 4 is a schematic diagram of a layered cluster tree. clonetype1 is the parent node of clonetype2 and clonetype3, clonetype4 and clonetype5 are child nodes of clonetype2, and clonetype6 is a child node of clonetype3.

A fusion genotype and sequence acquisition module is configured to align the DNA sequencing data to a reference gene sequence to acquire a fusion genotype and a corresponding sequence.

Specific steps include:

(1) Acquiring sequence counts of IGH, BCL1, and BCL2 separately using bwa alignment software.

(2) Constructing an FMDIndex data structure based on reference sequences of the IGH, BCL1, and BCL2 (https://pmc.ncbi.nlm.nih.gov/articles/PMC3389770/).

Finding a maximum set of exact match between each sequencing read and a corresponding reference sequence using FMDIndex.

Finding an optimal maximum subset of exact match between the reference sequence and the sequencing reads using a dynamic programming algorithm.

Aligning, using an edit distance algorithm, the sequencing read (seq1) to a reference gene sequence (seq2) corresponding to the optimal subset of exact match, so as to obtain a fusion type and a corresponding sequence count.

Acquiring a fusion type sequence by using a partial-order alignment algorithm based on a sequencing read corresponding to the fusion type.

The calculation module is configured to calculate a clonal frequency, a clonal cell abundance, a fusion frequency, and a fusion cell abundance based on the nucleated cell count, the clonotype profile and the corresponding sequence, and the fusion genotype and the corresponding sequence, and determine a dominant clonotype and a dominant fusion type based on the clonal frequency, the clonal cell abundance, the fusion frequency, and the fusion cell abundance.

Specifically, a calculation method for the clonal frequency and the clonal cell abundance is:

$$cfl = \frac{{count}_{1}}{{count}_{IGL}}$$

$$cfh = \frac{{count}_{2}}{sum ( {count}_{IGH} , {count}_{HDJ} )}$$

$$cfk = \frac{{count}_{3}}{sum ( {count}_{IGK} , {count}_{KDE} )}$$

$$clonal cell abundance = \frac{clonal cell count}{nucleated cell count}$$

In the formulas above, cfl represents a clonal frequency of a single IGL clonotype, count1 represents a sequence count of a single IGL clonotype, countIGL represents the sequence count of all IGL clonotypes, cfh represents a clonal frequency of a single IGH clonotype, count2 represents a sequence count of a single IGH clonotype, countIGH represents the sequence count of all IGH clonotypes except an IGH-DJ clonotype, countHDJ represents the sequence count of all IGH-DJ clonotypes; cfk represents a clonal frequency of a single IGK clonotype, count3 represents a sequence count of a single IGK clonotype, countIGK represents the sequence count of all IGK clonotypes except an IGK-KDE clonotype, countKDE represents the sequence count of all IGK-KDE clonotypes, and the clonal cell count is a sequence count of a single clonotype.

A calculation method for the fusion frequency and the fusion cell abundance is:

$$fusion frequency = max ( \frac{fc}{hc} , \frac{fc}{bc} )$$

$$fusion cell abundance = \frac{fusion cell count}{nucleated cell count}$$

In the formulas above, fc is a sequence count of a single fusion type, hc represents a total sequence count of IGH, bc is a total sequence count of BCL1 or BCL2, and the fusion cell count represents a sequence count of a single fusion type.

Preferably, a calculation method for the clonal frequency and the clonal cell abundance is:

$$cfl = \frac{{count}_{1}}{{count}_{IGL}} \times wf ( clonetype )$$

$$cfh = \frac{{count}_{2}}{sum ( {count}_{IGH} , {count}_{HDJ} )} \times wf ( clonetype )$$

$$cfk = \frac{{count}_{3}}{sum ( {count}_{IGK} , {count}_{KDE} )} \times wf ( clonetype )$$

$$clonal cell abundance = \frac{clonal cell count}{nucleated cell count} \times wf ( clonetype )$$

clonetype represents clonotype, and wf(clonetype) is a correction function.

A calculation method for the fusion frequency and the fusion cell abundance is:

$$fusion frequency = max ( \frac{fc}{hc} , \frac{fc}{bc} ) \times wf ( fusion )$$

$$fusion cell abundance = \frac{fusion cell count}{nucleated cell count} \times wf ( fusion )$$

fusion represents fusion type, and wf(fusion) is a correction function.

It needs to be noted that wf(clonetype) is a function related to the clonotype amplification efficiency and the clonotype amplification length; and wf(fusion) is a function related to the fusion type amplification efficiency and the fusion type amplification length.

A calculation method for the clonotype amplification efficiency is as follows:

First, a series of plasmids (that is, clonotypes) formed by various combinations of V and J regions of the IGH, IGK, and IGL are artificially synthesized; when IGH-DJ rearrangement occurs, plasmids formed by various combinations of D and J regions of the IGH are synthesized; when IGK-KDE rearrangement occurs, the KDE element and the J region of IGK are performed permutation and combination; these plasmids are dissolved and then quantified, and then are randomly mixed in equal mass to form 10 different plasmid pools. Each of the 10 plasmid pools is subjected to library preparation and sequencing. Analyze the data to generate a clonotype profile. For each plasmid, divide the read count by the average read count of the plasmids in the plasmid library, and record the result as value a. An amplification coefficient b (clonetype) is calculated based on a.

$$b ( clonetype ) = \{ \begin{matrix}\frac{1}{a} , if a \geq 0.1 and a \leq 10 \\ 0.1 , if a > 10 \\ 10 , if a > 0 and a \leq 0.1\end{matrix}$$

A calculation method for the fusion type amplification efficiency is as follows.

First, a series of plasmids (that is, fusion type) formed by combining IGH with BCL1 and BCL2 separately are artificially synthesized. These plasmids are dissolved and then quantified, and then are randomly mixed in equal mass to form 10 different plasmid pools. Each of the 10 plasmid pools is subjected to library preparation and sequencing. Analyze the data to obtain a fusion genotype. For each plasmid, divide the read count by the average read count of the plasmids in the plasmid library, and record the result as value a. An amplification coefficient b(fusion) is calculated based on a.

$$b ( fusion ) = \{ \begin{matrix}\frac{1}{a} , if a \geq 0.1 and a \leq 10 \\ 0.1 , if a > 10 \\ 10 , if a > 0 and a < 0.1\end{matrix}$$

After the amplification coefficient is obtained, a correction function is further calculated as follows based on the amplification length:

$$\{ \begin{matrix}wf ( clonetype ) = b ( clonetype ) * ( 1 + {λ}_{1} {e}^{- \frac{l ength ( plasmid )}{lengt h ( clonetype )}} ) \\ wf ( fusion ) = b ( fusion ) * ( 1 + {λ}_{2} {e}^{- \frac{l ength ( plasmid )}{l ength ( fusion )}} )\end{matrix}$$

In the formulas above, λ1 and λ2 are constant values. Preferably, λ1 may be 1.03 and λ2 may be 1.08. length (plasmid) is the length of the synthesized clonotype/fusion type (plasmid) sequence, length (clonetype) is the length of this clonotype sequence in a test sample, and length (fusion) is the length of this fusion type sequence in the test sample.

After the above data is obtained, the dominant clonotypes and the dominant fusion types are screened. The screening criteria for the dominant clonotypes are as follows:
(1) clonal frequency ≥0.03;
(2) clonal cell abundance ≥0.002; and
(3) clonal frequency distribution is discontinuous, as determined based on the following criteria:
(a) clonotypes with a frequency less than 0.03 are selected and arranged in descending order;
(b) 10 times a clonal frequency of a 5th clonotype arranged sequentially is used as a threshold; and
(c) clonal frequencies of all clonotypes are compared with the threshold, and a clonotype with a clonal frequency greater than or equal to the threshold is considered to satisfy discontinuity of clonal frequency distribution.

The dominant clonotype may be any one or more of IGH, IGK, IGL, IGH-DJ, or IGK-KDE, or a subset thereof.

Screening criteria for dominant fusion types are as follows:
(1) fusion frequency ≥0.1%; and
(2) fusion cell abundance ≥0.02%.

DNA samples are extracted from the same test subject before and after treatment separately to obtain pre-treatment dominant clonotypes and dominant fusion types as well as post-treatment clonotypes and fusion types separately. The treatment includes, but is not limited to, surgical treatment, immunotherapy, targeted therapy, chemotherapy, or radiotherapy.

Qualified clonotypes are selected based on the clonotypes obtained from the same test subject after treatment, and the clonal cell abundance of the qualified clonotypes is recorded, from which the MRDclone is calculated.

Specifically, qualified clonotypes include:
(1) clonotypes that exist in the same test subject after treatment and that are identical to the pre-treatment dominant clonotypes; and
(2) clonotypes that exist in the same test subject in subsequent tests and that are identical to the newly diagnosed post-treatment dominant clonotypes.

The newly diagnosed post-treatment dominant clonotypes refer to those dominant clonotypes that emerge after treatment and are not identical to any pre-treatment dominant clonotypes. In subsequent detections, the dominant clonotypes newly diagnosed after treatment are tracked.

The value of MRDclone is calculated by using one of the following methods, depending on whether the heavy chain (IGH) or the light chain (IGK, IGL) is tracked:
a. In the case that only heavy-chain (IGH) clonotypes are tracked

$$MRDclone = sum ( IGH clonal cell abundance )$$

b. In the case that only the light chain (IGK, IGL) clonotypes are tracked

$$MRDclone = max ( sum ( IGH clonal cell abundance ) , sum ( IGK clonal cell abundance ) + sum ( IGL clonal cell abundance ) )$$

c. In the case that both heavy chain clonotypes and light chain clonotypes are tracked

$$MRDclone = sum ( IGK clonal cell abundance ) + sum ( IGL clonal cell abundance )$$

This formula means selecting a maximum value among heavy chains and light chains to represent the MRDclone.

Qualified fusion types are selected based on the fusion types obtained from the same test subject after treatment, and the fusion cell abundance of the qualified fusion types is recorded, from which MRDfusion is calculated. The qualified fusion types are fusion types that exist in the same test subject after treatment and that are identical to the dominant fusion type existent before treatment.
a. In the case that only BCL1-IGH fusion types are tracked

$$MRDfusion = sum ( BCL 1 - IGH fusion cell abundance )$$

b. In the case that only BCL2-IGH fusion types are tracked

$$MRDfusion = sum ( BCL 2 - IGH fusion cell abundance )$$

c. In the case that both BCL1-IGH fusion types and BCL2-IGH fusion types are traced

$$MRDfusion = max ( sum ( BCL 1 - IGH fusion cell abundance ) , sum ( BCL 2 - IGH fusion cell abundance ) )$$

A calculation method for the MRD is:

$$MRDclone = max ( sum ( IGH clonal cell abundance ) , sum ( IGK clonal cell abundance ) + sum ( IGL clonal cell abundance ) )$$

$$MRDfusion = max ( sum ( BCL 1 - IGH fusion cell abundance ) , sum ( BCL 2 - IGH fusion cell abundance ) )$$

$$MRD = max ( {MRD}_{fusion} , {MRD}_{clone} )$$

$$or$$

$$MRD = {MRD}_{fusion} + {MRD}_{clone} .$$

When MRD is greater than 0, it is determined that residual hematologic malignancies exist; when MRD is equal to 0, it is determined that no residual hematologic malignancies exist.

For example, if dominant clonotype IGH-A (the clonal cell abundance is 3.64E-03) and IGH-B (the clonal cell abundance is 8.25E-03) are detected from a hematologic malignancy patient 1 before treatment, and clonotype IGH-A (the clonal cell abundance is 2.58E-06) is detected 3 months after treatment, then clonotype IGH-A is a qualified clonotype selected in this test. MRD=MRDclone=2.58E-06 >0. Therefore, the result of the test performed 3 months after treatment indicates existence of residual disease.

For example, if dominant fusion type BCL1-IGHJ (the fusion cell abundance is 5.79E-04) is detected from a hematologic malignancy patient 2 before treatment, and BCL1-IGHJ (the fusion cell abundance is 6.38E-06) is detected 3 months after treatment, then fusion type BCL1-IGHJ is a qualified fusion type selected in this test. MRD=MRDfusion=6.38E-06 >0. Therefore, the result of the test performed 3 months after treatment indicates existence of residual disease.

For example, if dominant clonotype IGH-A (the clonal cell abundance is 6.42E-02) and IGH-B (the clonal cell abundance is 4.12E-03) are detected from a hematologic malignancy patient 3 before treatment. No clonotypes identical to the dominant clonotypes IGH-A and IGH-B are detected 3 months after treatment, but a newly diagnosed dominant clonotype IGH-C(the clonal cell abundance is 5.35E-03) is detected. MRD=0. Therefore, the result of the test 3 months after treatment indicates no residual disease. This patient undergoes a new treatment regimen, and a test is performed 3 months after the new regimen is applied. The test results show that only clonotype IGH-C(the clonal cell abundance is 4.38E-04) is selected. Therefore, clonotype IGH-C is a qualified clonotype selected in this test. MRD=MRDclone=4.38E-04 >0. Therefore, the result of the test performed 3 months after the new regimen is applied indicates existence of residual disease.

The model further includes: acquiring clonotypes of healthy donors and/or patients with nonhomologous diseases, and calculating a clonal cell abundance of the clonotypes; and excluding, from dominant clonotypes obtained based on pre-treatment DNA sequencing data, those clonotypes that: (i) are non-rarely existent in the healthy donors and/or patients with nonhomologous diseases, and (ii) have a clonal cell abundance greater than or equal to 10-7. This step constitutes a basis for subsequent MRD calculation.

The above method may be run in the calculation module or in the MRD calculation and determination module. By acquiring clonotypes of healthy donors and/or patients with nonhomologous diseases, the clonotypes that are non-rarely existent and have a clonal cell abundance greater than or equal to 10-7 are excluded to obtain an optimal clonotype set. Furthermore, for the same test subject, a new dominant clonotype may arise after treatment, and the new dominant clonotype differs from the pre-treatment dominant clonotype, but the new dominant clonotype needs to be included into subsequent MRD calculations.

An embodiment of this application further provides a computer storage medium. The storage medium stores a computer program. When executed by a processor, the computer program implements the following method, including:
receiving pre-treatment and post-treatment DNA sequencing data of the same test subject;
aligning sequencing data of a housekeeping gene to a reference genome to acquire a nucleated cell count;
aligning the DNA sequencing data to an immune database using an alignment algorithm, and then acquiring a clonotype profile and a corresponding sequence using a clustering algorithm, where the immune database includes IGH, IGK, and IGL;
aligning the DNA sequencing data to a reference gene sequence to acquire a fusion genotype and a corresponding sequence;
calculating a clonal frequency, a clonal cell abundance, a fusion frequency, and a fusion cell abundance based on the nucleated cell count, the clonotype profile and the corresponding sequence, and the fusion genotype and the corresponding sequence, and determining a dominant clonotype and a dominant fusion type based on the clonal frequency, the clonal cell abundance, the fusion frequency, and the fusion cell abundance; and
selecting a qualified clonotype and a qualified fusion type based on clonotypes and fusion types obtained from the same subject before and after treatment, and calculating an MRD value based on the clonal cell abundance and the fusion cell abundance that correspond to the qualified clonotype and the qualified fusion type.

Further, the clustering algorithm includes: calculating an edit distance between sequences based on the DNA sequencing data; performing cluster analysis on sequencing reads based on the edit distance, the relationship between the edit distance and V, D, and J regions of IGH, the relationship between the edit distance and a V and J regions of IGK, and the relationship between the edit distance and V and J regions of IGL, so as to acquire a clonotype profile.

Further, a calculation method for the edit distance is:

$$editdist ( i , j ) = MAX \{ \begin{matrix}editdist ( i - 1 , j - 1 ) + m ( i , j ) \\ editdist ( i - 1 , j ) + gap ( i , j ) \\ editdist ( i , j - 1 ) + gap ( i , j ) \\ editdist ( i - 1 , j - 1 ) + S\end{matrix}$$

$$m ( i , j ) = \{ \begin{matrix}ms , & if seq {1}_{i} = seq {2}_{j} \\ ss , & if seq {1}_{i} \neq seq {2}_{j}\end{matrix}$$

$$gap ( i , j ) = \{ \begin{matrix}os , & if gap opening \\ es , & if gap extending\end{matrix}$$

In the formulas above, seq1 and seq2 represent 2 sequences, i represents the ith base in seq1, j represents the jth base in seq2, both i and j start from 1 up to the last base, and initial values thereof are all set to 0; ms represents a match score; when the ith base of seq1 matches the jth base of seq2, m(i, j) is a positive value; ss represents a mismatch score; when the ith base of seq1 does not match the jth base of seq2, m(i, j) is a negative value; os represents a gap-opening score; gap opening represents a start of a gap in sequence alignment; es represents a gap-extending score; gap extending represents continuation of a gap in sequence alignment; S represents a clip score; when the base of seql and the base of seq2 are so mismatched that a negative score is excessive, clipping is applied.

Further, the cluster analysis includes: determining an optimal alignment based on an alignment result and the edit distance, and clustering sequencing reads with identical V and J regions of IGH, IGK, and IGL; clustering sequencing reads with identical D and J regions of IGH when IGH-DJ rearrangement occurs; clustering sequencing reads with an identical J region of IGK and the KDE element when IGK-KDE rearrangement occurs; clustering sequencing reads within each VJ class based on consistency of CDR3 regions; clustering sequencing reads within each DJ class based on consistency of 50-bp regions centered on a D-J junction of IGH; clustering sequencing reads within each KDE-V class based on consistency of 50-bp regions centered on a junction between an IGK V region and a KDE element, to obtain a sequencing read count corresponding to each subclass; clustering each subclass by using a hierarchical clustering method, where a child node and a parent node within each cluster possess highly similar sequences, and the parent node possesses a significantly higher sequencing read count than the child node; summing sequencing read counts of all nodes in each cluster, and reporting a root-node sequence as a distinct clonotype; and combining all reported distinct clonotypes to form a clonotype profile.

Further, specifically, a calculation method for the clonal frequency and the clonal cell abundance is:

$$cfl = \frac{{count}_{1}}{c o u n {t}_{IGL}}$$

$$cfh = \frac{{count}_{2}}{s u m ( c o u n {t}_{IGH} , c o u n {t}_{HDJ} )}$$

$$cfk = \frac{{count}_{3}}{s u m ( c o u n {t}_{IGK} , c o u n {t}_{KDE} )}$$

$$clonal cell abundance = \frac{clonal cell count}{nucleated cell count}$$

In the formulas above, cfl represents a clonal frequency of a single IGL clonotype, count1 represents a sequence count of a single IGL clonotype, countIGL represents a sequence count of all IGL clonotypes, cfh represents a clonal frequency of a single IGH clonotype, count2 represents a sequence count of a single IGH clonotype, countIGH represents a sequence count of all IGH clonotypes except an IGH-DJ clonotype, countHDJ represents a sequence count of all IGH-DJ clonotypes; cfk represents a clonal frequency of a single IGK clonotype, count3 represents a sequence count of a single IGK clonotype, countIGK represents a sequence count of all IGK clonotypes except an IGK-KDE clonotype, countKDE represents a sequence count of all IGH-KDE clonotypes, and the clonal cell count is a sequence count of a single clonotype.

A calculation method for the fusion frequency and the fusion cell abundance is:

$$fusion frequency = max ( \frac{fc}{h c} , \frac{fc}{b c} )$$

$$fusion cell abundance = \frac{fusion cell count}{nucleated cell count}$$

In the formulas above, fc is a sequence count of a single fusion type, hc represents a total sequence count of IGH, bc is a total sequence count of BCL1 or BCL2, and the fusion cell count represents a sequence count of a single fusion type.

Preferably, a calculation method for the clonal frequency and the clonal cell abundance is:

$$cfl = \frac{c o u n {t}_{1}}{c o u n {t}_{IGL}} \times wf ( clonetype )$$

$$cfh = \frac{c o u n {t}_{2}}{sum ( c o u n {t}_{IGH} , {count}_{HDJ} )} \times wf ( clonetype )$$

$$cfk = \frac{{count}_{3}}{sum ( {count}_{IGK} , {count}_{KDE} )} \times wf ( clonetype )$$

$$clonal cell abundance = \frac{clonal cell count}{nucleated cell count} \times wf ( clonetype )$$

clonetype represents clonotype, and wf(clonetype) is a correction function.

A calculation method for the fusion frequency and the fusion cell abundance is:

$$fusion frequency = max ( \frac{fc}{h c} , \frac{fc}{b c} ) \times wf ( fusion )$$

$$fusion cell abundance = \frac{fusion cell count}{nucleated cell count} \times wf ( fusion )$$

fusion represents fusion type, and wf(fusion) is a correction function.

Further, screening criteria for dominant clonotypes are as follows:
(1) clonal frequency ≥0.03;
(2) clonal cell abundance ≥0.002; and
(3) clonal frequency distribution is discontinuous, as determined based on the following criteria:
(a) clonotypes with a frequency less than 0.03 are selected and arranged in descending order;
(b) 10 times a clonal frequency of a 5th clonotype arranged sequentially is used as a threshold; and
(c) clonal frequencies of all clonotypes are compared with the threshold, and a clonotype with a clonal frequency greater than or equal to the threshold is considered to satisfy discontinuity of clonal frequency distribution.

Further, screening criteria for dominant fusion types are as follows:
(1) fusion frequency ≥0.1%; and
(2) fusion cell abundance ≥0.02%.

Further, a calculation method for the MRD is:

MRDclone=max(sum(IGH clonal cell abundance), sum(IGK clonal cell abundance)+sum(IGL clonal cell abundance))

MRDfusion=max(sum(BCL1−IGH fusion cell abundance), sum(BCL2-IGH fusion cell abundance))

$$MRD = max ( {MRD}_{fusion} , {MRD}_{c l o n e} )$$

$$or$$

$$MRD = {MRD}_{fusion} + {MRD}_{c l o n e} .$$

Further, the model includes: acquiring clonotypes of healthy donors and/or patients with nonhomologous diseases, and calculating a clonal cell abundance of the clonotypes; and excluding, from dominant clonotypes obtained based on pre-treatment DNA sequencing data, those clonotypes that: (i) are non-rarely existent in the healthy donors and/or patients with nonhomologous diseases, and (ii) have a clonal cell abundance greater than or equal to 10-7, where the non-rarely existent clonotypes are clonotypes existent in 5% or more of a population.

### Embodiment 1: Detection of Minimal Residual Disease in Human Leukemia and Lymphoma

13 post-treatment samples with known pathological information are obtained from Genetron Health (Beijing) Co., Ltd. and its contracted hospitals. The cell line is purchased commercially. 4 of the samples are acute lymphoblastic leukemia (ALL) (samples 1 and 2 are bone marrow gDNA; samples 3 and 4 are peripheral blood gDNA). 4 of the samples are chronic lymphocytic leukemia (CLL) (samples 5 and 6 are bone marrow gDNA; samples 7 and 8 are peripheral blood gDNA). 3 of the samples are follicular lymphoma (FL) (samples 9, 10, and 11 are bone marrow gDNA). 2 of the samples are multiple myeloma (MM) (samples 12 and 13 are bone marrow gDNA). 1 of the samples is mantle cell lymphoma (MCL) (sample 14 is cell line gDNA).

Based on the technical solution disclosed in this application, MRD test results are obtained by using bone marrow DNA, peripheral blood DNA, or cell line DNA, which are extracted from the 13 patients before and after treatment, as starting samples, separately.

TABLE 7
Test results of samples

| Sample | | Clonotype/ | MRD |
|---|---|---|---|
| No. | Clone/fusion sequence | fusion type | value |
| 1 | TGTGCCCTCCGAGGGGCGAGAGATATTAGGCTG | IGH | 2.45E−03 |
| | GGATAGGTATTACTATGATAGTAGTGGTTGGACT | | |
| | GG | | |
| | ACAGTCGGGTATAGCAGCGCCCACAAGAGGCGT | IGH-DJ | |
| | CAGGGAACCCTGGTCAC | | |
| | AGTCGGGTATAGCAGCGCCCACAAGAGGCGTCA | IGH-DJ | |
| | GGGAACCCCGGTCACCG | | |
| | TATTACGTCTGCAGAGTAAGAATTTCCTAGCCCT | IGK-KDE | |
| | AGTGGCAGCCCAGGGC | | |
| 2 | TGTTCCAGAGCTAGGGGGGGGAGAGGGCCTAGG | IGH | 1.56E−05 |
| | GGAGAGGCAATCCGGTGGCCGTAGCAGCTGGTA | | |
| | CTGGTTCGACCCCTGG | | |
| | TGTCATCAGAGTAGTAGTTTACCCTCACTTTC | IGK | |
| | TGCTGCCGTAGCCAGCTTTCCCGTGGAGGGGGT | IGK-KDE | |
| | GGCAGCCCAGGGCGACT | | |
| 3 | TGTGCGAGGGATGGAGGAGTCTCCTTTAGTGGC | IGH | 6.01E−03 |
| | AGCCTTGACTACTGG | | |
| | TGCATGCAAGGTACACACTGGCCTCCGTTTAG | IGK | |
| | TGTCAGCAATATACTAATTGGCCTCCGATCACCTT | IGK | |
| | C | | |
| | GTCAACAGTATTATAGTTTCCCTCCTAGCCCAGG | IGK-KDE | |
| | GCGACTCCTCATGAGT | | |
| | ATGACCAGCAGATTCAAATCTATGGTGGTTTGAC | BCL2-IGHJ | |
| | CTTTAGAGAGTTGCTTTACGTGGCCTGTTTCAAC | fusion | |
| | ACAGACCCACCCAGAGCCCTCCTGCCCTCCTTC | | |
| | GGTTAACCCGGCCCCCAACTATAAACTTACTACT | | |
| | ACTACTACGGTATGGACGTCTGGGGCCAAGGGA | | |
| | CCAAGGTGGAAATCTCCTCAGGT | | |
| 4 | TGTGCACACAGGGGGGATACCCATACCCCTCGTG | IGH | 3.34E−04 |
| | GATATAGTGGCTACGATTGTAGGTGAGACTACTA | | |
| | CTACGGTATGGACGTCTGG | | |
| | TGTGCATAAGATTCCACGTGTAATGTGTATAGCA | IGH | |
| | GCAGCTGGTTCCCGCTTGATTAGGTACTACTACT | | |
| | ACGGTATGGACGTCTGG | | |
| 5 | TGTGCAAGAGCCCAAGAACCTCTATGGTCCAAC | IGH | 1.96E−02 |
| | TTTTACTACTACTACTACGGTATGGACGTCTGG | | |
| | TGTCAGCAGTATGGTAGCTCACTGTTTT | IGK | |
| | TGTCAACAGTATTATAGTACCTCACTTTC | IGK | |
| | TGCCTGCTCTACTATGGTGGTGCTCAGTGGGTGT | IGL | |
| | TC | | |
| | TCCTCCTACTGTGTGACTACAGTACCATGACTAC | IGH-DJ | |
| | TGGGGCCAGGGAACCC | | |
| | ATTAATGCTGCCGTAGCCAGCTTTCCAGCCCTAG | IGK-KDE | |
| | TGGCAGCCCAGGGCGA | | |
| 6 | TGTGCGCGAGCACCTGATGGCAGTGGTTACGAC | IGH | 4.32E−03 |
| | TACTTTGACTTCTGG | | |
| | TGTCAGCAGTATAATAGTTTGGTCACTTTC | IGK | |
| | CCTACTGTGTGACTCCAGTAACTACGGGCTGTGG | IGH-DJ | |
| | ACGTCTGGGGCAAGGG | | |
| 7 | TGTACGAAAGATATTAGCCAGAAGTCTCATCCGT | IGH | 3.49E−05 |
| | GGAGTGGGGACGGAATGGACGTCTGG | | |
| | TGTCAGCAATATAATAGTTCGTACAGTTTT | IGK | |
| | TGTCAACAGATTTTCAGTAATTCTCGGACTTTT | IGK | |
| | ATGACCAGCAGATTCAAATCTATGGTGGTTTGAC | BCL2-IGHJ | |
| | CTTTAGAGAGTTGCTTTACGTGGCCTGTTTCAAC | fusion | |
| | ACAGACCCACCCAGAGCCCTCCTGCCCTCCTTC | | |
| | CGCGGGGGCTTTCTCATGGCTGTCCTTCAGGGTC | | |
| | TTCCTGAAATGCAGTGGTGCTTACGGGGCCCTCG | | |
| | GGTTGGCTACTGGGGCCAGGGAACCCTGGTCAC | | |
| | CGTCTCCTCAGGT | | |
| 8 | TGTGCGAGGGGCGCGAATGGTTCGGGGAGTTAT | IGH | 9.81E−06 |
| | TTCGGGTACTACTTTGACCACTGG | | |
| | TGTGCGAGGGGCGCGAATGGTTCGGGGAGTTAT | IGH | |
| | TTCGGGTACTACTTCGACCCCTGG | | |
| | TGTCAGCAGCGTAGCAACTGGCCTCCTCTCACTT | IGK | |
| | TC | | |
| | TGCATGCAACGTATAGAGTTTCCTTCACTTTC | IGK | |
| | TGTCAGTCTTATGATAGTAACAATCATGTGGTCTT | IGL | |
| | C | | |
| | AACCCATGAGATTCATTCAGTTAAAACTGCAGAT | BCL2-IGHJ | |
| | GGCAAATGACCAGCAGATTCAAATATTTGGTGGT | fusion | |
| | TTGACCTTTGATTCCTAAACCCGGGACTACTACT | | |
| | ACTGCATGGACGTCTGGGGCCAAGGGCCCATGG | | |
| | TCACCGTCCCTTCGT | | |
| 9 | GGTCAACGGACTTACAATGCCCCTCGAACGTTC | IGK | 1.27E−05 |
| | TGTCAAAAGTATAACAGTGCCCCTCCCTTTC | IGK | |
| | TGTGCAACATGGGATGACAGTCTCAATGGTTACG | IGL | |
| | TGTTC | | |
| | TGTGCAACATGGGATGACAGTCTCAATGGTTACG | IGL | |
| | TCTTC | | |
| | GCTGCCGTAGCCAGCTTTCCTGATGACCTAGTGG | IGK-KDE | |
| | CAGCCCAGGGCGACTC | | |
| | ATTGATGCTGCCGTAGCCAGCTTTCCCTAGTGGC | IGK-KDE | |
| | AGCCCAGGGCGACTCC | | |
| | ATGACCAGCAGATTCAAATCTATGGTGGTTTGAC | BCL2-IGHJ | |
| | CTTTAGAGAGTTGCTTTACGTGGCCTGTTTCAAC | fusion | |
| | ACAGACCCACCCAGAGCCCTCCTGCCCTCCTTC | | |
| | CGCGGGGGCTTTCTCATGGCTGTCCTTCAGGGTC | | |
| | TTCCTGAAACGAGTTACTACTACTACTACTACAT | | |
| | GGACGTCTGGGGCAAAGGGACCACGGTCACCGT | | |
| | CTCCTCAGGT | | |
| 10 | GCATAGAGCAAGCGCCCAATAAATAGCAGCTATT | BCL2-IGHJ | 2.33−E03 |
| | GTTACTCTTGCAGGGTTCTTTAAGCAGCATAATA | fusion | |
| | GTCTTTAAATCATAAAATCTCAGCCTGCAGCGGG | | |
| | TAAATCCACTACTACTACTACTACATGGACGTCT | | |
| | GGGGCAAAGGGACCACGGTCACCGTCTCCTCAG | | |
| | GT | | |
| 11 | TGCATGCAAGGTACACACTGGCCTCCGACACTTT | IGK | 2.97E−03 |
| | C | | |
| 12 | TGCATGCAAGATGCACAAGATCGGGACTTTT | IGK | 7.91E−06 |
| | TGTCAGACCTGGGGCACTGGCTTTCATCAACAAT | IGL | |
| | TTTC | | |
| | TGCTTGCTCTCCTATACTGGTGCTCGGCCCGTGG | IGL | |
| | TATTC | | |
| | CTGTCAACAGAGTTACAGTACCCCTCGCAGCCC | IGK-KDE | |
| | AGGGCGACTCCTCATGA | | |
| 13 | TGTGCGAAAGACGACTCATTGGAATCTCGCGGG | IGH | 1.14E−04 |
| | CGGGCAAATTACTACATGGACGTCTGG | | |
| | TGTCAACAGAGTTACAGTACCCATTCACTTTC | IGK | |
| | TGAATGCAAGGTATACACCTTCGGGACACTTTT | IGK | |
| | TGCCAGTCCTATGACGAAAGCCTGCGTGGTTCG | IGL | |
| | AGGGTCTTC | | |
| | TGGTTCGGGGAGTTATTACCGCCCGTTCCTCTGG | IGH-DJ | |
| | CTACTGGGGCCAGGGA | | |
| 14 | TGTGCAAAAGATATGCAGGGAGCAGTGGCAGGG | IGH | 5.26E−05 |
| | GGCGTTGACTACTGG | | |
| | GGTCAACGGACTTACAATGCCCCTCCTCACTTTC | IGK | |
| | TGCAGCTCATATACAAGCAGCAGCGTGGTATTC | IGL | |
| | CTGTGATTAGCTTTCTAAAAAGTGGTTTTGTTAG | BCL1-IGHJ | |
| | ATGTAAAAAATGAATATGACATTCTGAAACAGAA | fusion | |
| | AAAAATAACTTACTCTTTATCTGAGTGGGATGAG | | |
| | ATTAAAGCCCCACTACTGGGGCCAGGGAACCCT | | |
| | GGTCACTGTCTCCTCAGGT | | |

Within the detection coverage of this test, all test results for different clonotypes ultimately show MRD-positive findings. As manifested above, using the detection method provided in this application to detect positive cases of human leukemia and lymphoma, MRD can be tracked and monitored effectively. In addition, the detection method exhibits good performance in terms of abundance of detectable clonotypes, thereby demonstrating high practical applicability of the detection method. Specifically, IGH-positive clonotypes are detected across four pathological types ALL, CLL, MM, and MCL, covering a total of 10 positive samples. IGK-positive clonotypes are detected across five pathological types ALL, CLL, FL, MM, and MCL, covering a total of 11 positive samples. IGL-positive clonotypes are detected across four pathological types CLL, FL, MM, and MCL, covering a total of 6 positive samples. HDJ-positive clonotypes are detected across three pathological types ALL, CLL, and MM, covering a total of 4 positive samples. KDE-positive clonotypes are detected across four pathological types ALL, CLL, FL, and MM, covering a total of 6 positive samples. Positive clonotypes for BCL-IGHJ rearrangement are detected across four pathological types ALL, CLL, FL, and MCL, covering a total of 6 positive samples. The test results show that this application enables effective detection of MRD in human leukemia, lymphoma, and myeloma.

### Embodiment 2: Consistency Evaluation in Sample Testing

A total of 21 samples are tested. 16 patients with known pathological information, including 6 patients with pre-treatment samples, 5 patients with post-treatment samples, and 5 patients with both pre-treatment samples and post-treatment samples. Sample 16 and sample 17, sample 18 and sample 19, sample 22 and sample 23, sample 25 and sample 26, and sample 28 and sample 29 are obtained from the same patient, and are subjected to clonotype testing by using the method of this application together with multicolor flow cytometry (MFC). The results of all 21 tests include 14 positive and 7 negative, which cover 11 pre-treatment samples and 10 post-treatment samples. The samples include 16 bone marrow samples and 5 peripheral blood samples, covering pathological types such as acute lymphoblastic leukemia (ALL), chronic lymphoblastic leukemia (CLL), and multiple myeloma (MM). The testing aims to evaluate the MRD detection performance of this application from a plurality of perspectives.

The specific results are shown in Table 8, where “NGS result” is a test result obtained using the method of this application, and “flow cytometry result” is a test result obtained using MFC. The test results show that the two methods yield consistent results across two sample types (bone marrow sample and peripheral blood sample) and three different pathological types, regardless of whether the samples are taken before or after treatment.

TABLE 8
Summary of test results of 21 clinical samples

| Sample | Sample | Sample | Disease | MFC | MFC | NGS | Consiste | Tracka |
|---|---|---|---|---|---|---|---|---|
| 15 | Before | Bone | ALL | 8.30E− | Positive | 9.36E− | a | Yes |
| | | | | [symbol] | | [symbol] | | |
| 16 | Before | Bone | ALL | 6.90E− | Positive | 3.10E− | a | Yes |
| | | | | [symbol] | | [symbol] | | |
| 17 | After | Bone | ALL | 1.22E− | Positive | 4.39E− | a | Yes |
| | | | | [symbol] | | [symbol] | | |
| 18 | Before | Bone | ALL | 7.30E− | Positive | 3.40E− | a | Yes |
| | | | | [symbol] | | [symbol] | | |
| 19 | After | Bone | ALL | 0 | Negativ | 0 | b | N/A |
| 20 | Before | Bone | CLL | 5.51E− | Positive | 6.83E− | a | Yes |
| | | | | [symbol] | | [symbol] | | |
| 21 | Before | Bone | CLL | 7.59E− | Positive | 9.31E− | a | Yes |
| | | | | [symbol] | | [symbol] | | |
| 22 | Before | Bone | CLL | 8.47E− | Positive | 6.33E− | a | Yes |
| | | | | [symbol] | | [symbol] | | |
| 23 | After | Bone | CLL | 4.98E− | Positive | 6.72E− | a | Yes |
| | | | | [symbol] | | [symbol] | | |
| 24 | Before | Bone | MM | 1.62E− | Positive | 4.14E− | a | Yes |
| | | | | [symbol] | | [symbol] | | |
| 25 | Before | Bone | MM | 7.29E− | Positive | 9.05E− | a | Yes |
| | | | | [symbol] | | [symbol] | | |
| 26 | After | Bone | MM | 1.35E− | Positive | 8.40E− | a | Yes |
| | | | | [symbol] | | [symbol] | | |
| 27 | Before | Peripheral | CLL | 4.11E− | Positive | 6.25E− | a | Yes |
| | | | | [symbol] | | [symbol] | | |
| 28 | Before | Peripheral | CLL | 7.60E− | Positive | 3.97E− | a | Yes |
| | | | | [symbol] | | [symbol] | | |
| 29 | After | Peripheral | CLL | 0 | Negativ | 0 | b | N/A |
| 30 | Before | Peripheral | ALL | 2.63E− | Positive | 1.02E− | a | Yes |
| | | | | [symbol] | | [symbol] | | |
| 31 | After | Bone | ALL | 0 | Negativ | 0 | c | No |
| 32 | After | Bone | ALL | 0 | Negativ | 0 | c | No |
| 33 | After | Bone | ALL | 0 | Negativ | 0 | b | N/A |
| 34 | After | Bone | ALL | 0 | Negativ | 0 | b | N/A |
| 35 | After | Peripheral | CLL | 0 | Negativ | 0 | b | N/A |

aBoth methods yield positive MRD results;
bBoth methods yield negative MRD results;
cBoth methods yield negative MRD results, and clonotypes are detected when the method of this application is applied, but the clonotypes are not disease-specific clonotypes due to existence in the clonotypes of healthy donors or nonhomologous patients, and therefore, are not tracked. [symbol]
indicates data missing or illegible when filed

### Embodiment 3: Sensitivity Study

1. Linear range achievable with an input amount of 20 μg DNA

The malignant-cell abundance is traced for an ALL cell line (RS4;11), a CLL cell line (MEC-1), and an MM cell line (IM-9) by using MFC. gDNA from tthree cell lines is diluted with gDNA isolated from leukocytes of whole blood samples from healthy donors, based on the traced malignant-cell frequency. The initial number of cells subjected to detection was calculated according to the input amount of nucleic acid (20 μg). Linear reference standards are prepared with the malignant-cell abundance concentrations set at 1E-06, 1E-05, 1E-04, 1E-03, and 1E-02 respectively. Cell number=starting DNA input amount (in pg)/6.6 μg per cell. The prepared serially diluted sample is tested 3 times repeatedly by using the method of this application to investigate the detectable linear range of this method.

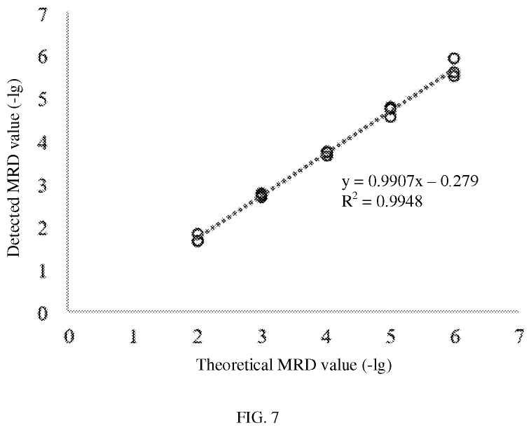

At an input amount of 20 μg DNA, the disease types are ALL, CLL, and MM, and the detection range corresponds to the above gradient reference materials. The theoretical frequency and the MRD value are converted to-lg values for statistical analysis separately. The data is shown in Table 9 and FIG. 5 to FIG. 7. The dilution linearity of the 3 disease-related cell lines shows R2≥0.95, demonstrating that the test method of this application provides high linearity when the DNA input amount is 20 μg.

TABLE 9
Linear relationship results at an input amount of 20 μg DNA

| Disease | Linear range | Slope | Intercept | Correlation coefficient | R2 |
|---|---|---|---|---|---|
| ALL | 1E−06 to 1E−02 | 0.99 | −0.65 | 0.997 | 0.994 |
| CLL | 1E−06 to 1E−02 | 0.98 | −0.08 | 0.997 | 0.994 |
| MM | 1E−06 to 1E−02 | 0.99 | −0.28 | 0.997 | 0.995 |

2. Establishment of limit of detection (LOD) at an input amount of 20 μg DNA

Trace the origin of malignant-cell abundance in the ALL cell line (RS4;11), CLL cell line (MEC-1), and MM cell line (IM-9) using MFC. Dilute the genomic DNA (gDNA) of these three cell lines with the gDNA of leukocytes from healthy human whole blood according to the traced malignant cell frequency, and adjust the dilution to the target malignant cell frequency required for the limit of detection (LOD) verification as specified in Table 10.

TABLE 10
Correspondence between malignant-cell frequency and
malignant-cell count at an input amount of 20 μg DNA
3076923 cells in total

| Malignant-cell count | Frequency |
|---|---|
| 2 | 6.50E−07 |
| 3 | 9.75E−07 |
| 4 | 1.30E−06 |

Note:
The DNA mass here is calculated based on the number of bases in the human genome and the diploid characteristics, assuming that 1 cell contains 6.5 pg of DNA.

At an input amount of 20 μg DNA, the test is performed 3 times repeatedly at 3 MRD frequencies separately using the method of this application, so as to establish a preliminary LOD at a 100% detection frequency. As can be seen from the results in Table 11, the lowest frequency at which positive results are detected three times in all 3 disease types is 6.50E-07.

TABLE 11
Detection results of LOD establishment at an input amount of 20 μg DNA

| | ALL | | | CLL | | | MM | | |
|---|---|---|---|---|---|---|---|---|---|
| Theoretical | 6.50E− | 9.75E− | 1.30E− | 6.50E− | 9.75E− | 1.30E− | 6.50E− | 9.75E− | 1.30E− |
| frequency | 07 | 07 | 06 | 07 | 07 | 06 | 07 | 07 | 06 |
| 1 | 1.72E− | 1.39E− | 2.63E− | 1.19E− | 2.05E− | 2.74E− | 1.05E− | 1.51E− | 2.23E− |
| | 06 | 05 | 06 | 06 | 06 | 06 | 06 | 06 | 06 |
| 2 | 1.64E− | 6.99E− | 4.93E− | 7.64E− | 1.34E− | 2.83E− | 1.00E− | 1.95E− | 3.48E− |
| | 06 | 06 | 06 | 07 | 06 | 06 | 06 | 06 | 06 |
| 3 | 6.49E− | 3.36E− | 6.31E− | 9.94E− | 1.55E− | 3.49E− | 5.39E− | 1.85E− | 1.66E− |
| | 07 | 06 | 06 | 07 | 06 | 06 | 07 | 06 | 06 |
| Mean | 1.34E− | 8.08E− | 4.63E− | 9.82E− | 1.65E− | 3.02E− | 8.63E− | 1.77E− | 2.46E− |
| value | 06 | 06 | 06 | 07 | 06 | 06 | 07 | 06 | 06 |
| Positive | 100% | 100% | 100% | 100% | 100% | 100% | 100% | 100% | 100% |
| detection | | | | | | | | | |
| rate | | | | | | | | | |

3. Confirmation of LOD at an input amount of 20 μg DNA

The ALL, CLL, and MM cell lines are selected as reference materials for establishing an LOD. The gDNA of the three cell lines is diluted to a very low malignant-cell count of 2 or 3 malignant cells separately. At an input amount of 20 μg DNA, the samples are tested in 13 replicates using the method of this application. The LOD is defined as the lowest malignant-cell abundance at which a 100% positive detection rate is still achieved. Based on the results in Table 12, the LOD determined for ALL, CLL, and MM using the method of this application at a DNA input of 20 μg is 6.50E-07.

TABLE 12
Results of confirmed LOD at an input of 20 μg

| ALL | | | CLL | | | MM | | |
|---|---|---|---|---|---|---|---|---|
| Replicate | | | Replicate | | | Replicate | | |
| number | MRD value | | number | MRD value | | number | MRD value | |
| 1 | 1.94E− | 1.32E− | 1 | 2.05E− | 1.19E− | 1 | 1.93E− | 1.34E− |
| | 06 | 06 | | 06 | 06 | | 06 | 06 |
| 2 | 1.06E− | 4.72E− | 2 | 1.34E− | 7.64E− | 2 | 3.18E− | 1.48E− |
| | 06 | 06 | | 06 | 07 | | 06 | 06 |
| 3 | 5.72E− | 5.43E− | 3 | 1.55E− | 9.94E− | 3 | 2.45E− | 1.39E− |
| | 06 | 06 | | 06 | 07 | | 06 | 06 |
| 4 | 1.48E− | 2.21E− | 4 | 8.00E− | 6.03E− | 4 | 3.45E− | 2.46E− |
| | 06 | 06 | | 06 | 07 | | 06 | 06 |
| 5 | 2.04E− | 1.25E− | 5 | 1.59E− | 1.05E− | 5 | 6.10E− | 2.91E− |
| | 06 | 06 | | 06 | 06 | | 07 | 06 |
| 6 | 1.12E− | 2.40E− | 6 | 1.22E− | 2.80E− | 6 | 2.42E− | 1.65E− |
| | 06 | 06 | | 06 | 06 | | 06 | 06 |
| 7 | 6.51E− | 1.58E− | 7 | 2.08E− | 7.58E− | 7 | 1.74E− | 2.06E− |
| | 06 | 06 | | 06 | 07 | | 06 | 06 |
| 8 | 6.16E− | 1.83E− | 8 | 2.14E− | 2.44E− | 8 | 5.13E− | 3.17E− |
| | 06 | 06 | | 06 | 06 | | 07 | 06 |
| 9 | 3.91E− | 2.31E− | 9 | 1.61E− | 1.20E− | 9 | 1.49E− | 8.00E− |
| | 06 | 06 | | 06 | 06 | | 06 | 07 |
| 10 | 4.84E− | 1.32E− | 10 | 1.41E− | 2.35E− | 10 | 3.22E− | 1.53E− |
| | 06 | 06 | | 06 | 06 | | 06 | 06 |
| 11 | 2.00E− | 3.26E− | 11 | 1.73E− | 1.07E− | 11 | 7.16E− | 1.60E− |
| | 06 | 07 | | 06 | 06 | | 06 | 06 |
| 12 | 3.44E− | 6.82E− | 12 | 1.80E− | 7.72E− | 12 | 2.94E− | 2.75E− |
| | 06 | 07 | | 06 | 07 | | 06 | 06 |
| 13 | 6.36E− | 1.12E− | 13 | 2.67E− | 5.44E− | 13 | 1.44E− | 3.78E− |
| | 06 | 06 | | 06 | 06 | | 06 | 07 |
| Mean | 3.58E− | 2.04E− | Mean | 2.20E− | 1.60E− | Mean | 2.50E− | 1.81E− |
| value | 06 | 06 | value | 06 | 06 | value | 06 | 06 |
| Theoretical | 9.75E− | 6.50E− | Theoretical | 9.75E− | 6.50E− | Theoretical | 9.75E− | 6.50E− |
| value | 07 | 07 | value | 07 | 07 | value | 07 | 07 |

### Embodiment 4: CtDNA Detection

Based on the previously validated sample types, this application further extends the sample types to ctDNA. Plasma samples from 8 patients with known pathological diagnoses are selected, including 3 cases of diffuse large B-cell lymphoma (DLBCL), 1 case of mantle cell lymphoma (MCL), 1 case of primary central nervous system lymphoma (PCNSL), and 3 cases of multiple myeloma (MM). The ctDNA extracted from pre-treatment and post-treatment samples of each patient is subjected to MRD detection using the method described in Embodiment 1. The detection results are shown in Table 13.

TABLE 13
Detection results of ctDNA sample

| Sam | Disease | Clonot | Clonal sequence | MRD |
|---|---|---|---|---|
| 1 | DLBC | IGK | TGTCAGCAATATAATAAGTGGCCGTCCACTTTT | 4.11E− |
| 2 | DLBCL | IGH | TGTGCGAGAGATGGTTACGATTTTTGGAGTGGTT | 1.71E−03 |
| | | IGK | TGTCAAAAGTATAACAGTGCCCCTATCACTTTC | |
| | | IGK | TGTCAGCAGTATGGTAGCTCACCTTACACTTTT | |
| 3 | DLBCL | IGH | TGTGCCAGAGGGTCCCTGGTTTCATTCGAAGCTG | 2.20E−03 |
| | | IGK | TGTCAACAGTATGATAATCTCCCTCCTCGCTCACT | |
| | | IGK | TGCATGCAAGCTCTACAAACCCCACGTGGACGTT | |
| 4 | MCL | IGH | TGTGCGAGAGATCTTTGGGTATACAACGGTCCATT | 2.17E−04 |
| | | IGK | TGTCAGCAGTATAATAACTGGCCTCCGGGTTCCG | |
| 5 | PCNSL | IGK | TGTCAGCAATATTATAGTACTCCGTTCACTTTT | 2.01E− |
| 6 | MM | IGK | TGCCAACAATATGATAGTTTTCCATTCACTTTC | 4.09E− |
| 7 | MM | IGH | TGTGCGAGAGGGTCCATTTCGGACAACTATGACT | 1.95E−02 |
| | | IGK | TGCCAACAGTATCATACTAATTCGACGTTC | |
| | | IGK | TGCATGCAAGATGCACAAGATCCTCCGAACACTT | |
| 8 | MM | IGH | TGTGTACGCCGCTATTGTACTGGTGCTGACTGCTA | 8.65E−05 |
| | | IGK | TGTCAGCAGTATGGTAACTCACAGTGGACGTTC | |

### Embodiment 5: Detection of Fusions

Fusion genes are one of the earliest identified genetic abnormalities in hematologic malignancies and have clear significance for clinical diagnosis and treatment. The latest World Health Organization (WHO) 2016 classification of tumors of hematopoietic and lymphoid tissues explicitly lists 112 types of fusion genes (FGs), most of which can serve as a basis for diagnosis and classification. The fusion genes included herein for tracing are BCL1−IGH and BCL2-IGH.

Sanger sequencing is performed to analyze traceability of the frequency of the BCL1-IGH fusion gene in the JVM-2 cell line. According to the traced malignant cell frequencies sorted in descending order, the gDNA of the JVM-2 cell line was serially diluted with the gDNA of leukocytes isolated from healthy human whole blood to the target frequencies specified in Table 14 below. At an input amount of 20 μg DNA, each frequency standard sample is tested in triplicate to preliminarily establish the LOD.

TABLE 14
Correspondence between malignant-cell frequency and
malignant-cell number at different DNA input amounts
20 μg
3076923 cells in total

| Malignant-cell number | Frequency |
|---|---|
| 5 | 1.63E−06 |
| 10 | 3.25E−06 |
| 15 | 4.88E−06 |

For 3 different DNA input amounts (disease type: MCL), each frequency standard sample is tested in triplicate, and the LOD is preliminarily established at a 100% detection frequency.

TABLE 15
Detection results of LOD establishment at
an input amount of 20 μg DNA

| Theoretical frequency | 1.63E−06 | 3.25E−06 | 4.88E−06 |
|---|---|---|---|
| 1 | 5.27E−06 | 3.66E−06 | 6.88E−06 |
| 2 | 2.05E−06 | 7.44E−06 | 1.16E−05 |
| 3 | 1.87E−06 | 8.63E−06 | 7.38E−06 |
| Mean value | 3.06E−06 | 6.58E−06 | 8.61E−06 |
| SD | 1.91E−06 | 2.59E−06 | 2.58E−06 |
| CV | 62% | 39% | 30% |
| Positive detection rate | 100% | 100% | 100% |

### Embodiment 6: Trackable Positive Clonotypes, that is, Clonotype Cases Existent Both Before and After Treatment

4 patients with known pathological information are selected. Samples are collected at different treatment time points. MRD detection is performed using the method of this application. The detection results are shown in Table 16. For patient 1, 2 dominant clonotypes are detected in the pre-treatment sample. Follow-up samples collected 3 months after treatment show no recurrence of the 2 dominant clonotypes. Samples collected 6 months after treatment show no residual of the 2 pre-treatment dominant clonotypes, but show a new clonotype with a clonal cell abundance of 5.43E-03. For patient 2, 3 dominant clonotypes are detected before treatment. Residual clonotypes at a frequency of 10-6 are detected 3 months after treatment. 6 months after treatment, a rebound occurs and residual clonotypes are detected at a high concentration. For patient 3, 3 dominant clonotypes are detected before treatment. 3 months after treatment, the 3 dominant clonotypes are also detected but at a reduced clonal cell abundance. For patient 4, 2 dominant clonotypes are detected before treatment. 3 months after treatment, the clonal cell abundance of one clonotype decreases to 10-6, and no residual IGH-A clonotype is detected. For patients 3 and 4, no data is collected 6 months after treatment.

TABLE 16
MRD monitoring results

| | | | | | Clonal cell | Clonal cell |
|---|---|---|---|---|---|---|
| | | | | Clonal cell | abundance | abundancet |
| | | | | abundance | 3 months | 6 months |
| Patient | Sample | Disease | Clono- | before | after | after |
| No. | type | type | type | treatment | treatment | treatment |
| 1 | Bone | B-ALL | IGH-A | 2.22E−01 | 0.00E+00 | 0.00E+00 |
| | marrow | | IGK-B | 2.24E−01 | 0.00E+00 | 0.00E+00 |
| | | | IGH-C | — | — | 5.43E−03 |
| | | | (new) | | | |
| 2 | Bone | B-ALL | IGH-A | 3.29E−01 | 3.78E−06 | 3.60E−01 |
| | marrow | | IGK-B | 5.70E−01 | 5.79E−06 | 6.57E−01 |
| | | | IGK-C | 1.74E−01 | 1.51E−06 | 1.77E−01 |
| 3 | Bone | B-ALL | IGH-A | 1.96E−01 | 7.85E−03 | — |
| | marrow | | IGH-B | 4.04E−02 | 1.82E−03 | — |
| | | | IGH-C | 8.63E−03 | 2.92E−04 | — |
| 4 | Bone | B-ALL | IGH-A | 4.05E−03 | 0.00E+00 | — |
| | marrow | | IGK-B | 2.44E−03 | 8.58E−06 | — |

Described above are merely preferred embodiments of this application that are not intended to limit this application. Any modifications, equivalent substitutions, and improvements made without departing from the concept and principles of this application still fall within the protection scope of this application.

1. A model for monitoring minimal residual disease in human hematologic malignancies, comprising:
a data acquisition module, configured to receive pre-treatment and post-treatment DNA sequencing data of the same test subject;
a nucleated cell count acquisition module, configured to align sequencing data of a housekeeping gene to a reference genome to acquire a nucleated cell count;
a clonotype profile and sequence acquisition module, configured to align the DNA sequencing data to an immune database using an alignment algorithm, and then acquire a clonotype profile and a corresponding sequence using a clustering algorithm, wherein the immune database comprises IGH, IGK, and IGL;
a fusion genotype and sequence acquisition module, configured to align the DNA sequencing data to a reference gene sequence to acquire a fusion genotype and a corresponding sequence;
a calculation module, configured to calculate a clonal frequency, a clonal cell abundance, a fusion frequency, and a fusion cell abundance based on the nucleated cell count, the clonotype profile and the corresponding sequence, and the fusion genotype and the corresponding sequence, and determine a dominant clonotype and a dominant fusion type based on the clonal frequency, the clonal cell abundance, the fusion frequency, and the fusion cell abundance; and
an MRD calculation and determination module, configured to screen a qualified clonotype and a qualified fusion type based on clonotypes and fusion types obtained from the same subject before and after treatment, calculate an MRD value based on a clonal cell abundance and a fusion cell abundance that correspond to the qualified clonotype and the qualified fusion type, and determine residual status of a hematologic malignancy based on the MRD value, wherein
the clustering algorithm comprises:
calculating an edit distance between sequences based on the DNA sequencing data;
performing cluster analysis on sequencing reads based on the edit distance, the relationship between the edit distance and V, D, and J regions of IGH, the relationship between the edit distance and V and J regions of IGK, and the relationship between the edit distance and V and J regions of IGL, so as to acquire a clonotype profile;
a calculation method for the edit distance is:
$$editdist ( i , j ) = MAX \{ \begin{matrix}editdist ( i − 1 , j − 1 ) + m ( i , j ) \\ editdist ( i − 1 , j ) + gap ( i , j ) \\ editdist ( i , j − 1 ) + gap ( i , j ) \\ editdist ( i − 1 , j − 1 ) + S\end{matrix} ;$$
$$m ( i , j ) = \{ \begin{matrix}ms , if seq {1}_{i} = seq {2}_{j} \\ ss , if seq {1}_{i} \neq seq {2}_{j}\end{matrix} ;$$
$$gap ( i , j ) = \{ \begin{matrix}o s , if gap extending \\ es , if gap extending\end{matrix} ;$$
wherein, seq1 and seq2 represent 2 sequences, i represents the ith base in seq1, j represents the jth base in seq2, both i and j start from 1 up to a last base, and initial values thereof are all set to 0; ms represents a match score; when the ith base of seq1 matches the jth base of seq2, m(i, j) is a positive value; ss represents a mismatch score; when the ith base of seq1 does not match the jth base of seq2, m(i, j) is a negative value; os represents a gap-opening score; gap opening represents a start of a gap in sequence alignment; es represents a gap-extending score; gap extending represents continuation of a gap in sequence alignment; S represents a clip score; when the base of seq1 and the base of seq2 are so mismatched that a negative score is excessive, clipping is applied;
values of ms, ss, os, es, and S satisfy the following formula:
$$\{ \begin{matrix}m s > 0 \\ s s < 0 \\ o s < 0 \\ e s < 0 \\ s < 0 \\ \vert S \vert < \vert os \vert + n ⋆ \vert es \vert + 2 \\ \vert os \vert > \vert ss \vert \geq \vert es \vert\end{matrix} ;$$
wherein, n is an integer greater than or equal to 0 and less than a sequencing read length.

2. The model for monitoring minimal residual disease in human hematologic malignancies according to claim 1, wherein the cluster analysis comprises:
determining an optimal alignment based on an alignment result and the edit distance, and clustering sequencing reads with identical V and J regions of IGH, IGK, and IGL; clustering sequencing reads with identical D and J regions of IGH when IGH-DJ rearrangement occurs; and clustering sequencing reads with an identical V region of the IGK and the KDE element when IGK-KDE rearrangement occurs;
clustering sequencing reads within each VJ class based on consistency of CDR3 regions; clustering sequencing reads within each DJ class based on consistency of 50-bp regions centered on a D-J junction of IGH; and clustering sequencing reads within each KDE-V class based on consistency of 50-bp regions centered on a junction between an IGK V region and a KDE element, to obtain a sequencing read count corresponding to each subclass;
clustering each subclass using a hierarchical clustering method, wherein a child node and a parent node within each cluster possess highly similar sequences, and the parent node possesses a significantly higher sequencing read count than the child node;
summing sequencing read counts of all nodes in each cluster, and reporting a root-node sequence as a distinct clonotype; and
combining all reported distinct clonotypes to form a clonotype profile.

3. The model for monitoring minimal residual disease in human hematologic malignancies according to claim 1, wherein the method for the fusion genotype and sequence acquisition module to acquire the fusion genotype and the corresponding sequence comprises:
acquiring sequence counts of IGH, BCL1, and BCL2 separately using bwa alignment software;
constructing an FMDIndex data structure based on reference sequences of IGH, BCL1, and BCL2;
finding a maximum set of exact match between each sequencing read and a corresponding reference sequence using FMDIndex;
finding an optimal maximum subset of exact match between the reference sequence and the sequencing read using a dynamic programming algorithm;
aligning, using an edit distance algorithm, the sequencing read to a reference gene sequence corresponding to the optimal subset of exact match, so as to obtain a fusion genotype and a corresponding sequence count; and
acquiring a fusion genotype sequence using a partial-order alignment algorithm based on a sequencing read corresponding to the fusion genotype.

4. The model for monitoring minimal residual disease in human hematologic malignancies according to claim 1, wherein
a calculation method for the clonal frequency and the clonal cell abundance is:
$$cfl = \frac{c o u n {t}_{1}}{c o u n {t}_{IGL}} ;$$
$$cfh = \frac{c o u n {t}_{2}}{sum ( c o u n {t}_{IGH} , {count}_{HDJ} )} ;$$
$$cfk = \frac{{count}_{3}}{sum ( {count}_{IGK} , {count}_{KDE} )} ;$$
$$clonal cell abundance = \frac{clonal cell count}{nucleated cell count} ;$$
wherein, cfl represents a clonal frequency of a single IGL clonotype, count1 represents a sequence count of a single IGL clonotype, countIGL represents a sequence count of all IGL clonotypes, cfh represents a clonal frequency of a single IGH clonotype, count2 represents a sequence count of a single IGH clonotype, countIGH represents a sequence count of all IGH clonotypes except an IGH-DJ clonotype, countHDJ represents a sequence count of all IGH-DJ clonotypes; cfk represents a clonal frequency of a single IGK clonotype, count3 represents a sequence count of a single IGK clonotype, countIGK represents a sequence count of all IGK clonotypes except an IGK-KDE clonotype, countKDE represents a sequence count of all IGK-KDE clonotypes, and the clonal cell count is a sequence count of a single clonotype.

5. The model for monitoring minimal residual disease in human hematologic malignancies according to claim 1, wherein
a calculation method for the clonal frequency and the clonal cell abundance is:
$$cfl = \frac{c o u n {t}_{1}}{c o u n {t}_{IGL}} \times wf ( clonetype ) ;$$
$$cfh = \frac{c o u n {t}_{2}}{sum ( c o u n {t}_{IGH} , {count}_{HDJ} )} \times wf ( clonetype ) ;$$
$$cfk = \frac{{count}_{3}}{sum ( {count}_{IGK} , {count}_{KDE} )} \times wf ( clonetype ) ;$$
$$clonal cell abundance = \frac{clonal cell count}{nucleated cell count} \times wf ( clonetype ) ;$$
wherein, clonetype represents clonotype, and wf(clonetype) is a correction function;
$$wf = ( clonetype ) = b ( clonetype ) * ( 1 + {λ}_{1} {e}^{- \frac{length ( plasmid )}{length ( clonestype )}} ) ;$$
wherein, λ1 is 1.03, length (plasmid) is a length of a synthesized clonotype plasmid sequence, and length (clonetype) is a length of this clonotype sequence in a test sample;
$$b ( clonetype ) = \{ \begin{matrix}\frac{1}{a} , if a \geq 0.1 and a \leq 10 \\ 0.1 , if a > 10 \\ 10 , if a > 0 and a < 0.1\end{matrix} ;$$
firstly, a series of plasmids formed by various combinations of V and J regions of IGH, IGK, and IGL are artificially synthesized; when IGH-DJ rearrangement occurs, plasmids formed by various combinations of D and J regions of IGH are synthesized; when IGK-KDE rearrangement occurs, the KDE element and the J region of IGK are performed permutation and combination; these plasmids are dissolved and then quantified, and then are randomly mixed in equal mass to form 10 different plasmid pools; each of the 10 plasmid pools is subjected to library preparation and sequencing; analyze the data to generate a clonotype profile; for each plasmid, divide the read count by the average read count of the plasmids in the plasmid library, and record the result as value a.

6. The model for monitoring minimal residual disease in human hematologic malignancies according to claim 1, wherein
a calculation method for the fusion frequency and the fusion cell abundance is:
$$fusion frequency = max ( \frac{fc}{h c} , \frac{fc}{b c} ) ;$$
$$fusion cell abundance = \frac{fusion cell count}{nucleated cell count} ;$$
wherein, fc is a sequence count of a single fusion type, hc represents a total sequence count of IGH, bc is a total sequence count of BCL1 or BCL2, and the fusion cell count represents a sequence count of a single fusion type.

7. The model for monitoring minimal residual disease in human hematologic malignancies according to claim 1, wherein
a calculation method for the fusion frequency and the fusion cell abundance is:
$$fusion frequency = max ( \frac{fc}{h c} , \frac{fc}{b c} ) \times wf ( fusion ) ;$$
$$fusion cell abundance = \frac{fusion cell count}{nucleated cell count} \times wf ( fusion ) ;$$
wherein, fusion represents fusion type, and wf(fusion) is a correction function;
$$wf ( fusion ) = b ( fusion ) * ( 1 + {λ}_{2} {e}^{- \frac{length ( plasmid )}{length ( fusion )}} ) ;$$
wherein, λ2 is 1.08, length (plasmid) is a length of a synthesized fusion plasmid sequence, and length (fusion) is a length of this fusion sequence in a test sample;
$$b ( fusion ) = \{ \begin{matrix}\frac{1}{a} , if a \geq 0.1 and a \leq 10 \\ 0.1 , if a > 10 \\ 10 , if a > 0 and a < 0.1\end{matrix} ;$$
firstly, a series of plasmids formed by combining the IGH gene with the BCLI gene and the BCL2 gene separately are artificially synthesized; these plasmids are dissolved and then quantified, and then are randomly mixed in equal mass to form 10 different plasmid pools; each of the 10 plasmid pools is subjected to library preparation and sequencing; analyze the data to obtain a fusion genotype; for each plasmid, divide the read count by the average read count of the plasmids in the plasmid library, and record the result as value a.

8. The model for monitoring minimal residual disease in human hematologic malignancies according to claim 1, wherein
screening criteria for dominant clonotypes are as follows:
(1) clonal frequency ≥0.03;
(2) clonal cell abundance ≥0.002; and
(3) clonal frequency distribution being discontinuous, as determined based on the following criteria:
(a) clonotypes with a frequency less than 0.03 being selected and arranged in descending order;
(b) 10 times a clonal frequency of a 5th clonotype arranged sequentially being used as a threshold; and
(c) clonal frequencies of all clonotypes being compared with the threshold, and a clonotype with a clonal frequency greater than or equal to the threshold being considered to satisfy discontinuity of clonal frequency distribution.

9. The model for monitoring minimal residual disease in human hematologic malignancies according to claim 1, wherein screening criteria for dominant fusion types are as follows:
(1) fusion frequency ≥0.1%; and
(2) fusion cell abundance ≥0.02%.

10. The model for monitoring minimal residual disease in human hematologic malignancies according to claim 1, wherein
a calculation method for the MRD is:
$$MRDclone = max ( sum ( IGH clonal cell abundance ) , sum ( IGK clonal cell abundance ) + sum ( IGL clonal cell abundance ) ) ;$$
$$MRDfusion = max ( sum ( BCL 1 - IGH fusion cell abundance ) , sum ( BCL 2 - IGH fusion cell abundance ) ) ;$$
$$MRD = max ( {MRD}_{fusion} , {MRD}_{c l o n e} ;$$
$$or$$
$$MRD = {MRD}_{fusion} + {MRD}_{c l o n e} .$$

11. The model for monitoring minimal residual disease in human hematologic malignancies according to claim 1, wherein the model further comprises:
acquiring clonotypes of healthy donors and/or patients with nonhomologous diseases, and calculating a clonal cell abundance of the clonotypes; and
excluding, based on the clonotypes of the healthy donors and/or patients with nonhomologous diseases, from dominant clonotypes obtained based on pre-treatment DNA sequencing data, clonotypes that are non-rarely existent in the healthy donors and/or patients with nonhomologous diseases and that have a clonal cell abundance greater than or equal to 10-7, wherein the non-rarely existent clonotypes are clonotypes existent in 5% or more of a population.


SEQUENCE LISTING (185 sequences)

SEQ ID NO: 1 - DNA, 31 bp, synthetic construct
  source 1..31: mol_type = other DNA
ggcctacaattcaaattaatgtaaaaactgc

SEQ ID NO: 2 - DNA, 28 bp, synthetic construct
  source 1..28: mol_type = other DNA
tctgattcaaacttttcttttgtggctg

SEQ ID NO: 3 - DNA, 28 bp, synthetic construct
  source 1..28: mol_type = other DNA
gcactttcaatcaactgtgttagattga

SEQ ID NO: 4 - DNA, 29 bp, synthetic construct
  source 1..29: mol_type = other DNA
tatgatccacattgtatggtttttaggca

SEQ ID NO: 5 - DNA, 24 bp, synthetic construct
  source 1..24: mol_type = other DNA
caagcagaagacggcatacgagat

SEQ ID NO: 6 - DNA, 10 bp, synthetic construct
  source 1..10: mol_type = other DNA
taggtccgat

SEQ ID NO: 7 - DNA, 10 bp, synthetic construct
  source 1..10: mol_type = other DNA
ggacggaatc

SEQ ID NO: 8 - DNA, 10 bp, synthetic construct
  source 1..10: mol_type = other DNA
cttactgccg

SEQ ID NO: 9 - DNA, 10 bp, synthetic construct
  source 1..10: mol_type = other DNA
acctaattga

SEQ ID NO: 10 - DNA, 10 bp, synthetic construct
  source 1..10: mol_type = other DNA
ttcgtatccg

SEQ ID NO: 11 - DNA, 10 bp, synthetic construct
  source 1..10: mol_type = other DNA
ggtaacgagc

SEQ ID NO: 12 - DNA, 10 bp, synthetic construct
  source 1..10: mol_type = other DNA
caacgtataa

SEQ ID NO: 13 - DNA, 10 bp, synthetic construct
  source 1..10: mol_type = other DNA
acgtcgcgtt

SEQ ID NO: 14 - DNA, 10 bp, synthetic construct
  source 1..10: mol_type = other DNA
ttctgctagc

SEQ ID NO: 15 - DNA, 10 bp, synthetic construct
  source 1..10: mol_type = other DNA
aggaagatag

SEQ ID NO: 16 - DNA, 17 bp, synthetic construct
  source 1..17: mol_type = other DNA
ggcacccgagaattcca

SEQ ID NO: 17 - DNA, 17 bp, synthetic construct
  source 1..17: mol_type = other DNA
cgaaccgctttcggagg

SEQ ID NO: 18 - DNA, 20 bp, synthetic construct
  source 1..20: mol_type = other DNA
aagggcttgagtggatggga

SEQ ID NO: 19 - DNA, 20 bp, synthetic construct
  source 1..20: mol_type = other DNA
aacatggaccctgtggacac

SEQ ID NO: 20 - DNA, 21 bp, synthetic construct
  source 1..21: mol_type = other DNA
acagcctacatggagctgagc

SEQ ID NO: 21 - DNA, 21 bp, synthetic construct
  source 1..21: mol_type = other DNA
accttgaaggagtctggtcct

SEQ ID NO: 22 - DNA, 20 bp, synthetic construct
  source 1..20: mol_type = other DNA
agtcaccatgaccagggaca

SEQ ID NO: 23 - DNA, 17 bp, synthetic construct
  source 1..17: mol_type = other DNA
tgcagctgcaggagtcg

SEQ ID NO: 24 - DNA, 20 bp, synthetic construct
  source 1..20: mol_type = other DNA
ggaaaggcytggagtggatg

SEQ ID NO: 25 - DNA, 20 bp, synthetic construct
  source 1..20: mol_type = other DNA
ccctgaagctgagctctgtg

SEQ ID NO: 26 - DNA, 18 bp, synthetic construct
  source 1..18: mol_type = other DNA
ggtgaagccctcgcagac

SEQ ID NO: 27 - DNA, 20 bp, synthetic construct
  source 1..20: mol_type = other DNA
ccatcgagaggccttgagtg

SEQ ID NO: 28 - DNA, 25 bp, synthetic construct
  source 1..25: mol_type = other DNA
aagtcgaataaccatcaacccagac

SEQ ID NO: 29 - DNA, 21 bp, synthetic construct
  source 1..21: mol_type = other DNA
ccctgagactctcctgtgcag

SEQ ID NO: 30 - DNA, 22 bp, synthetic construct
  source 1..22: mol_type = other DNA
cggggagtctctgargatctcc

SEQ ID NO: 31 - DNA, 17 bp, synthetic construct
  source 1..17: mol_type = other DNA
ctgcagtggagcagcct

SEQ ID NO: 32 - DNA, 22 bp, synthetic construct
  source 1..22: mol_type = other DNA
ccgattcaccatctccagagac

SEQ ID NO: 33 - DNA, 17 bp, synthetic construct
  source 1..17: mol_type = other DNA
atgcactgggtccgcca

SEQ ID NO: 34 - DNA, 19 bp, synthetic construct
  source 1..19: mol_type = other DNA
cttggtccagcaggcttca

SEQ ID NO: 35 - DNA, 17 bp, synthetic construct
  source 1..17: mol_type = other DNA
agggaaggggctggagt

SEQ ID NO: 36 - DNA, 19 bp, synthetic construct
  source 1..19: mol_type = other DNA
ccagggaacagactggagt

SEQ ID NO: 37 - DNA, 20 bp, synthetic construct
  source 1..20: mol_type = other DNA
ctgggaaagggctggagtag

SEQ ID NO: 38 - DNA, 23 bp, synthetic construct
  source 1..23: mol_type = other DNA
cttcacaacaggaattggtgtgg

SEQ ID NO: 39 - DNA, 23 bp, synthetic construct
  source 1..23: mol_type = other DNA
cttcacaacagggataggtgtgg

SEQ ID NO: 40 - DNA, 19 bp, synthetic construct
  source 1..19: mol_type = other DNA
ccagggaagggaatggagt

SEQ ID NO: 41 - DNA, 20 bp, synthetic construct
  source 1..20: mol_type = other DNA
tctgggagggaatggagtgg

SEQ ID NO: 42 - DNA, 18 bp, synthetic construct
  source 1..18: mol_type = other DNA
ggcaacaggctggaatgg

SEQ ID NO: 43 - DNA, 19 bp, synthetic construct
  source 1..19: mol_type = other DNA
aaggaagggactggagcga

SEQ ID NO: 44 - DNA, 21 bp, synthetic construct
  source 1..21: mol_type = other DNA
ccagtcctcagtcaagggagt

SEQ ID NO: 45 - DNA, 21 bp, synthetic construct
  source 1..21: mol_type = other DNA
ccagggaagaagctgaagtgg

SEQ ID NO: 46 - DNA, 20 bp, synthetic construct
  source 1..20: mol_type = other DNA
gcttcaagaaaggggctgga

SEQ ID NO: 47 - DNA, 20 bp, synthetic construct
  source 1..20: mol_type = other DNA
cctggatccaccatctccca

SEQ ID NO: 48 - DNA, 18 bp, synthetic construct
  source 1..18: mol_type = other DNA
ggcaaggggctggagtga

SEQ ID NO: 49 - DNA, 22 bp, synthetic construct
  source 1..22: mol_type = other DNA
gcttgcccctagaagatggaca

SEQ ID NO: 50 - DNA, 16 bp, synthetic construct
  source 1..16: mol_type = other DNA
gaaggcgctggggcac

SEQ ID NO: 51 - DNA, 18 bp, synthetic construct
  source 1..18: mol_type = other DNA
gggacagaggctggagtg

SEQ ID NO: 52 - DNA, 22 bp, synthetic construct
  source 1..22: mol_type = other DNA
ctgtaggcactggatggaaagc

SEQ ID NO: 53 - DNA, 20 bp, synthetic construct
  source 1..20: mol_type = other DNA
catgggaggtactggagtgg

SEQ ID NO: 54 - DNA, 19 bp, synthetic construct
  source 1..19: mol_type = other DNA
cagggaaggggctggaatg

SEQ ID NO: 55 - DNA, 19 bp, synthetic construct
  source 1..19: mol_type = other DNA
cagggaatgggctggagtt

SEQ ID NO: 56 - DNA, 19 bp, synthetic construct
  source 1..19: mol_type = other DNA
aggaaaggggctgcagtga

SEQ ID NO: 57 - DNA, 18 bp, synthetic construct
  source 1..18: mol_type = other DNA
aggagaggggctggagtg

SEQ ID NO: 58 - DNA, 22 bp, synthetic construct
  source 1..22: mol_type = other DNA
ttcatggcaagtgttggtgagg

SEQ ID NO: 59 - DNA, 22 bp, synthetic construct
  source 1..22: mol_type = other DNA
ctcagtgaaggtctcctgcaag

SEQ ID NO: 60 - DNA, 23 bp, synthetic construct
  source 1..23: mol_type = other DNA
cttctgggaaagggctggagtgg

SEQ ID NO: 61 - DNA, 20 bp, synthetic construct
  source 1..20: mol_type = other DNA
ttcaggacaggggttggtgt

SEQ ID NO: 62 - DNA, 21 bp, synthetic construct
  source 1..21: mol_type = other DNA
cctggacagggctttgagtgg

SEQ ID NO: 63 - DNA, 22 bp, synthetic construct
  source 1..22: mol_type = other DNA
ccatggacaagggcttgaggaa

SEQ ID NO: 64 - DNA, 17 bp, synthetic construct
  source 1..17: mol_type = other DNA
atgacaggggtgggtgt

SEQ ID NO: 65 - DNA, 19 bp, synthetic construct
  source 1..19: mol_type = other DNA
ccagggaagggactggagt

SEQ ID NO: 66 - DNA, 22 bp, synthetic construct
  source 1..22: mol_type = other DNA
aatgagactctagggaaggggc

SEQ ID NO: 67 - DNA, 20 bp, synthetic construct
  source 1..20: mol_type = other DNA
cacatgggaaggactggagt

SEQ ID NO: 68 - DNA, 18 bp, synthetic construct
  source 1..18: mol_type = other DNA
actagcccgcttccatgg

SEQ ID NO: 69 - DNA, 23 bp, synthetic construct
  source 1..23: mol_type = other DNA
ctccaggaaagggtttggagtgg

SEQ ID NO: 70 - DNA, 19 bp, synthetic construct
  source 1..19: mol_type = other DNA
gaaggccctggagtggctt

SEQ ID NO: 71 - DNA, 22 bp, synthetic construct
  source 1..22: mol_type = other DNA
ccctggacaagggcttgaaagg

SEQ ID NO: 72 - DNA, 22 bp, synthetic construct
  source 1..22: mol_type = other DNA
gggctggagtgggtaggtttca

SEQ ID NO: 73 - DNA, 24 bp, synthetic construct
  source 1..24: mol_type = other DNA
ggaagggtctggagtgggtatcag

SEQ ID NO: 74 - DNA, 20 bp, synthetic construct
  source 1..20: mol_type = other DNA
aaggggctggagtgggtatc

SEQ ID NO: 75 - DNA, 23 bp, synthetic construct
  source 1..23: mol_type = other DNA
gagcagcctgagatctgaggact

SEQ ID NO: 76 - DNA, 22 bp, synthetic construct
  source 1..22: mol_type = other DNA
ccgmttcatcatctccagagac

SEQ ID NO: 77 - DNA, 23 bp, synthetic construct
  source 1..23: mol_type = other DNA
gattcygaacagccccgagtcac

SEQ ID NO: 78 - DNA, 23 bp, synthetic construct
  source 1..23: mol_type = other DNA
ggattttgtgggggytcgtgtca

SEQ ID NO: 79 - DNA, 22 bp, synthetic construct
  source 1..22: mol_type = other DNA
ggtaggtttgrrgtgaggtctg

SEQ ID NO: 80 - DNA, 23 bp, synthetic construct
  source 1..23: mol_type = other DNA
gccggtaggtttagaatgaggtc

SEQ ID NO: 81 - DNA, 24 bp, synthetic construct
  source 1..24: mol_type = other DNA
ctttttgtgaaggsccctcctrct

SEQ ID NO: 82 - DNA, 23 bp, synthetic construct
  source 1..23: mol_type = other DNA
cactcaggggctttttgtgaagg

SEQ ID NO: 83 - DNA, 21 bp, synthetic construct
  source 1..21: mol_type = other DNA
gcctccagttccaggtgtggt

SEQ ID NO: 84 - DNA, 24 bp, synthetic construct
  source 1..24: mol_type = other DNA
ggtttctgaagstgtctgtrtcac

SEQ ID NO: 85 - DNA, 26 bp, synthetic construct
  source 1..26: mol_type = other DNA
ccaggagacaaagtcaacatctcctg

SEQ ID NO: 86 - DNA, 25 bp, synthetic construct
  source 1..25: mol_type = other DNA
cctcacaattaatcctgtggaagct

SEQ ID NO: 87 - DNA, 17 bp, synthetic construct
  source 1..17: mol_type = other DNA
agagccggcctccatct

SEQ ID NO: 88 - DNA, 21 bp, synthetic construct
  source 1..21: mol_type = other DNA
agtggggtcccatcaaggttc

SEQ ID NO: 89 - DNA, 22 bp, synthetic construct
  source 1..22: mol_type = other DNA
gggaaagagtcaccctctcctg

SEQ ID NO: 90 - DNA, 22 bp, synthetic construct
  source 1..22: mol_type = other DNA
ctggagtcccagacaggttcag

SEQ ID NO: 91 - DNA, 25 bp, synthetic construct
  source 1..25: mol_type = other DNA
cagatttcactctcaccatcagcag

SEQ ID NO: 92 - DNA, 23 bp, synthetic construct
  source 1..23: mol_type = other DNA
ggagagccatcctccatttcctg

SEQ ID NO: 93 - DNA, 21 bp, synthetic construct
  source 1..21: mol_type = other DNA
ggaaagagccaccctctcctg

SEQ ID NO: 94 - DNA, 24 bp, synthetic construct
  source 1..24: mol_type = other DNA
ggccagtcagagcattggtagtag

SEQ ID NO: 95 - DNA, 23 bp, synthetic construct
  source 1..23: mol_type = other DNA
cccagacagattcagtggcagtg

SEQ ID NO: 96 - DNA, 25 bp, synthetic construct
  source 1..25: mol_type = other DNA
tggagaaaaaagccaccctgacttg

SEQ ID NO: 97 - DNA, 24 bp, synthetic construct
  source 1..24: mol_type = other DNA
ctggagtgccagataggttcagtg

SEQ ID NO: 98 - DNA, 22 bp, synthetic construct
  source 1..22: mol_type = other DNA
ggttcagtggaagtggatctgg

SEQ ID NO: 99 - DNA, 20 bp, synthetic construct
  source 1..20: mol_type = other DNA
gaaggagccaccctcacctg

SEQ ID NO: 100 - DNA, 23 bp, synthetic construct
  source 1..23: mol_type = other DNA
ggagacagtgtcaccatcacttg

SEQ ID NO: 101 - DNA, 20 bp, synthetic construct
  source 1..20: mol_type = other DNA
ccatctcggttcagtggcag

SEQ ID NO: 102 - DNA, 24 bp, synthetic construct
  source 1..24: mol_type = other DNA
tggagtcccagacaggttcaatag

SEQ ID NO: 103 - DNA, 22 bp, synthetic construct
  source 1..22: mol_type = other DNA
gagacagcgtcaccatcacttg

SEQ ID NO: 104 - DNA, 25 bp, synthetic construct
  source 1..25: mol_type = other DNA
actgcatctgtaggagagagagtca

SEQ ID NO: 105 - DNA, 23 bp, synthetic construct
  source 1..23: mol_type = other DNA
gagaaagtcaccatcacctgcca

SEQ ID NO: 106 - DNA, 20 bp, synthetic construct
  source 1..20: mol_type = other DNA
ctggagagccagcctccatc

SEQ ID NO: 107 - DNA, 24 bp, synthetic construct
  source 1..24: mol_type = other DNA
tgcatctgtaggagacagagtcag

SEQ ID NO: 108 - DNA, 19 bp, synthetic construct
  source 1..19: mol_type = other DNA
caggcagggcattagccat

SEQ ID NO: 109 - DNA, 22 bp, synthetic construct
  source 1..22: mol_type = other DNA
aggttcaggggtaatgggtcag

SEQ ID NO: 110 - DNA, 27 bp, synthetic construct
  source 1..27: mol_type = other DNA
tctttatctctaggggaaaaagccacc

SEQ ID NO: 111 - DNA, 20 bp, synthetic construct
  source 1..20: mol_type = other DNA
tcccacctcgattcagtggc

SEQ ID NO: 112 - DNA, 22 bp, synthetic construct
  source 1..22: mol_type = other DNA
ggttcagtggaagtgggtctgg

SEQ ID NO: 113 - DNA, 21 bp, synthetic construct
  source 1..21: mol_type = other DNA
cagctgcagactcatgaggag

SEQ ID NO: 114 - DNA, 21 bp, synthetic construct
  source 1..21: mol_type = other DNA
ggaccgtgggacagaaggtca

SEQ ID NO: 115 - DNA, 21 bp, synthetic construct
  source 1..21: mol_type = other DNA
gcttctcaggctccagctctg

SEQ ID NO: 116 - DNA, 18 bp, synthetic construct
  source 1..18: mol_type = other DNA
tgtggggcaggtgggtac

SEQ ID NO: 117 - DNA, 24 bp, synthetic construct
  source 1..24: mol_type = other DNA
cctctctgcacatctgagaaatgc

SEQ ID NO: 118 - DNA, 23 bp, synthetic construct
  source 1..23: mol_type = other DNA
ggagcatgagccagacttacctg

SEQ ID NO: 119 - DNA, 21 bp, synthetic construct
  source 1..21: mol_type = other DNA
cggatcagccagactctcctg

SEQ ID NO: 120 - DNA, 19 bp, synthetic construct
  source 1..19: mol_type = other DNA
ggagcaacagccagactcc

SEQ ID NO: 121 - DNA, 20 bp, synthetic construct
  source 1..20: mol_type = other DNA
gtcaccatctcctgcactgg

SEQ ID NO: 122 - DNA, 20 bp, synthetic construct
  source 1..20: mol_type = other DNA
ccacaaggcagaggctcact

SEQ ID NO: 123 - DNA, 23 bp, synthetic construct
  source 1..23: mol_type = other DNA
ggagggacagtcacactcacttg

SEQ ID NO: 124 - DNA, 19 bp, synthetic construct
  source 1..19: mol_type = other DNA
cctcggtcaagctcacctg

SEQ ID NO: 125 - DNA, 23 bp, synthetic construct
  source 1..23: mol_type = other DNA
ctctgggagcatcttccagactc

SEQ ID NO: 126 - DNA, 22 bp, synthetic construct
  source 1..22: mol_type = other DNA
gagcctcggtcaagcttacctc

SEQ ID NO: 127 - DNA, 26 bp, synthetic construct
  source 1..26: mol_type = other DNA
ctcaccatctctggactgaagactga

SEQ ID NO: 128 - DNA, 21 bp, synthetic construct
  source 1..21: mol_type = other DNA
cttgagacagaccgccacact

SEQ ID NO: 129 - DNA, 22 bp, synthetic construct
  source 1..22: mol_type = other DNA
ccgcttctctggatccaaagat

SEQ ID NO: 130 - DNA, 24 bp, synthetic construct
  source 1..24: mol_type = other DNA
tctgcatcttctggagcatcagtc

SEQ ID NO: 131 - DNA, 21 bp, synthetic construct
  source 1..21: mol_type = other DNA
ttctctggctccaagtctggc

SEQ ID NO: 132 - DNA, 23 bp, synthetic construct
  source 1..23: mol_type = other DNA
atccctgatcgcttctcagtctt

SEQ ID NO: 133 - DNA, 20 bp, synthetic construct
  source 1..20: mol_type = other DNA
ctcactgactgtgtccccag

SEQ ID NO: 134 - DNA, 20 bp, synthetic construct
  source 1..20: mol_type = other DNA
tgtctgtggccttgggacaa

SEQ ID NO: 135 - DNA, 21 bp, synthetic construct
  source 1..21: mol_type = other DNA
acagacasccaggatcacctg

SEQ ID NO: 136 - DNA, 23 bp, synthetic construct
  source 1..23: mol_type = other DNA
aggacagatgactaggatcacct

SEQ ID NO: 137 - DNA, 22 bp, synthetic construct
  source 1..22: mol_type = other DNA
tcctggacagtcgatcaccatc

SEQ ID NO: 138 - DNA, 21 bp, synthetic construct
  source 1..21: mol_type = other DNA
gtccctaggacagatggccag

SEQ ID NO: 139 - DNA, 20 bp, synthetic construct
  source 1..20: mol_type = other DNA
acagatggccaggatcacct

SEQ ID NO: 140 - DNA, 20 bp, synthetic construct
  source 1..20: mol_type = other DNA
ccaggacaaacggccaggat

SEQ ID NO: 141 - DNA, 22 bp, synthetic construct
  source 1..22: mol_type = other DNA
gacagagggccaggatcaccta

SEQ ID NO: 142 - DNA, 21 bp, synthetic construct
  source 1..21: mol_type = other DNA
acaggcagccatgatcacctg

SEQ ID NO: 143 - DNA, 22 bp, synthetic construct
  source 1..22: mol_type = other DNA
ggtctcctggacagtcagtcac

SEQ ID NO: 144 - DNA, 21 bp, synthetic construct
  source 1..21: mol_type = other DNA
ctgggagcctcgatcaagctc

SEQ ID NO: 145 - DNA, 21 bp, synthetic construct
  source 1..21: mol_type = other DNA
ggacgtacggccaggatcatc

SEQ ID NO: 146 - DNA, 20 bp, synthetic construct
  source 1..20: mol_type = other DNA
aggacagacagccagcatca

SEQ ID NO: 147 - DNA, 21 bp, synthetic construct
  source 1..21: mol_type = other DNA
acagtcagccagggtaacctg

SEQ ID NO: 148 - DNA, 19 bp, synthetic construct
  source 1..19: mol_type = other DNA
aggacactcactcccccac

SEQ ID NO: 149 - DNA, 24 bp, synthetic construct
  source 1..24: mol_type = other DNA
cttgggacagacagtcaggatcac

SEQ ID NO: 150 - DNA, 19 bp, synthetic construct
  source 1..19: mol_type = other DNA
gcctccctggccatctctg

SEQ ID NO: 151 - DNA, 22 bp, synthetic construct
  source 1..22: mol_type = other DNA
cgtctgtctgtggctaaaccag

SEQ ID NO: 152 - DNA, 22 bp, synthetic construct
  source 1..22: mol_type = other DNA
atggcatctctgaccatctctg

SEQ ID NO: 153 - DNA, 22 bp, synthetic construct
  source 1..22: mol_type = other DNA
ggatccctgagcgattctctgg

SEQ ID NO: 154 - DNA, 19 bp, synthetic construct
  source 1..19: mol_type = other DNA
cactgtctggatgcaccgc

SEQ ID NO: 155 - DNA, 25 bp, synthetic construct
  source 1..25: mol_type = other DNA
atgaccagcagattcaaatctatgg

SEQ ID NO: 156 - DNA, 25 bp, synthetic construct
  source 1..25: mol_type = other DNA
ggaataactctgtggcattattgca

SEQ ID NO: 157 - DNA, 22 bp, synthetic construct
  source 1..22: mol_type = other DNA
tgagcacctgctggatacaaca

SEQ ID NO: 158 - DNA, 24 bp, synthetic construct
  source 1..24: mol_type = other DNA
ctgggtgacagagcaaaacatgaa

SEQ ID NO: 159 - DNA, 25 bp, synthetic construct
  source 1..25: mol_type = other DNA
ctgtaatgactggggagcaaatctt

SEQ ID NO: 160 - DNA, 21 bp, synthetic construct
  source 1..21: mol_type = other DNA
gttggcgtggtttagagagct

SEQ ID NO: 161 - DNA, 24 bp, synthetic construct
  source 1..24: mol_type = other DNA
tcccttctgaaagaaacgaaagca

SEQ ID NO: 162 - DNA, 25 bp, synthetic construct
  source 1..25: mol_type = other DNA
gcatagagcaagcgcccaataaata

SEQ ID NO: 163 - DNA, 27 bp, synthetic construct
  source 1..27: mol_type = other DNA
cagaaatgaatgccatctcaaatccaa

SEQ ID NO: 164 - DNA, 19 bp, synthetic construct
  source 1..19: mol_type = other DNA
acctgaggagacggtgacc

SEQ ID NO: 165 - DNA, 20 bp, synthetic construct
  source 1..20: mol_type = other DNA
gaggagacagtgaccagggt

SEQ ID NO: 166 - DNA, 22 bp, synthetic construct
  source 1..22: mol_type = other DNA
ctgaagagacggtgaccattgt

SEQ ID NO: 167 - DNA, 18 bp, synthetic construct
  source 1..18: mol_type = other DNA
atgtggctgcggtctcca

SEQ ID NO: 168 - DNA, 19 bp, synthetic construct
  source 1..19: mol_type = other DNA
tgacgttcccagggagacg

SEQ ID NO: 169 - DNA, 20 bp, synthetic construct
  source 1..20: mol_type = other DNA
ctgacccctcagaagccaga

SEQ ID NO: 170 - DNA, 26 bp, synthetic construct
  source 1..26: mol_type = other DNA
acttacgtttaatctccagtcgtgtc

SEQ ID NO: 171 - DNA, 23 bp, synthetic construct
  source 1..23: mol_type = other DNA
cgtttgatctccaccttggtccc

SEQ ID NO: 172 - DNA, 24 bp, synthetic construct
  source 1..24: mol_type = other DNA
acgtttgatatccactttggtccc

SEQ ID NO: 173 - DNA, 20 bp, synthetic construct
  source 1..20: mol_type = other DNA
gatctccagcttggtcccct

SEQ ID NO: 174 - DNA, 22 bp, synthetic construct
  source 1..22: mol_type = other DNA
atttccaccttggtcccttggc

SEQ ID NO: 175 - DNA, 23 bp, synthetic construct
  source 1..23: mol_type = other DNA
cgtttgaattctaccctgctccc

SEQ ID NO: 176 - DNA, 20 bp, synthetic construct
  source 1..20: mol_type = other DNA
gatstccagtttggtcccct

SEQ ID NO: 177 - DNA, 17 bp, synthetic construct
  source 1..17: mol_type = other DNA
ggctttggtggccatgc

SEQ ID NO: 178 - DNA, 21 bp, synthetic construct
  source 1..21: mol_type = other DNA
ctaggacggtgaccttggtcc

SEQ ID NO: 179 - DNA, 20 bp, synthetic construct
  source 1..20: mol_type = other DNA
cggtcagcttggtccctccg

SEQ ID NO: 180 - DNA, 22 bp, synthetic construct
  source 1..22: mol_type = other DNA
atgatcagctgggttcctccac

SEQ ID NO: 181 - DNA, 19 bp, synthetic construct
  source 1..19: mol_type = other DNA
aggacggtcaccttggtgc

SEQ ID NO: 182 - DNA, 17 bp, synthetic construct
  source 1..17: mol_type = other DNA
aggacggtcagctgggt

SEQ ID NO: 183 - DNA, 23 bp, synthetic construct
  source 1..23: mol_type = other DNA
agactcatctaggacggtcagct

SEQ ID NO: 184 - DNA, 24 bp, synthetic construct
  source 1..24: mol_type = other DNA
tcagagttctacagtccgacgatc

SEQ ID NO: 185 - DNA, 31 bp, synthetic construct
  source 1..31: mol_type = other DNA
aatgatacggcgaccaccgagatctacacgt